(19) United States
(12) Patent Application Publication
(10) Pub. No.: US 2025/0049510 A1
(43) Pub. Date: Feb. 13, 2025

(54) SYSTEMS AND METHODS FOR DIGITAL CONTROL OF ENDOVASCULAR DEVICES

(71) Applicant: RAPID MEDICAL LTD., Yokneam (IL)

(72) Inventors: Ronen ECKHOUSE, Yokneam (IL); Giora KORNBLAU, Yokneam (IL); Yuval VAKNIN, Yokneam (IL)

(73) Assignee: RAPID MEDICAL LTD., Yokneam (IL)

(21) Appl. No.: 18/719,614

(22) PCT Filed: Dec. 13, 2022
(86) PCT No.: PCT/IB2022/062141

§ 371 Date: Jun. 13, 2024

Related U.S. Application Data
(60) Provisional application No. 63/265,331, filed on Dec. 13, 2021.
(60) Provisional application No. 63/381,286, filed on Oct. 27, 2022.
(60) Provisional application No. 63/422,074, filed on Nov. 3, 2022.

(51) Int. Cl. A61B 34/20; A61B 1/008; A61B 8/00; A61B 17/00; A61B 17/221; A61B 90/00

(52) U.S. Cl. CPC A61B 34/20; A61B 1/008; A61B 8/54; A61B 17/221; A61B 2017/00221; A61B 2017/00398; A61B 2034/2059; A61B 2090/064; A61B 2090/374; A61B 2090/3762; A61B 2090/378

(57) ABSTRACT

Consistent with disclosed embodiments, systems, devices, methods, and computer readable media for digital control of an endovascular device and for controlling movement of an endovascular device may be provided. Embodiments may include a control device configured to be positioned outside a body of a patient. Embodiments may also include an input mechanism configured to receive input from a user. Embodiments may also include a device movement mechanism configured to control at least one movable portion of the endovascular device, the movable portion of the endovascular device configured for placement within the body of the patient.

### CROSS-REFERENCE TO RELATED APPLICATIONS

This application claims the benefit of priority of U.S. Provisional Application No. 63/265,331, filed Dec. 13, 2021; U.S. Provisional Application No. 63/381,286, filed Oct. 27, 2022; and U.S. Provisional Application No. 63/422,074, filed Nov. 3, 2022, each of which is hereby incorporated by reference in its entirety.

### TECHNICAL FIELD AND BACKGROUND

The present disclosure relates to systems, methods, and computer readable media for digital control of endovascular devices. For example, disclosed embodiments may be configured to digitally control an endovascular device to execute at least one desired action within a body structure of a patient.

Endovascular procedures, such as aneurysm embolization or the treatment of blood clots with expandable mesh devices, are important tools in today's treatment of cardiovascular and neurovascular disease. Endovascular procedures are highly complex operations that are often performed in narrow, tortuous vasculature. But despite this complexity, most endovascular procedures are performed manually, with the physician mechanically operating an endovascular device with precise maneuvers to achieve the desired actions by the portions of the endovascular device positioned at the treatment site. To add to this complexity, the same maneuvers by the physician may cause different results when performed with different endovascular devices, when used in patients with different characteristics, or when treating different sizes and types of anatomy. Mistakes in the forces applied by the physician, either due to a slip of a hand, to an error in judgment, or to lack of experience with a specific endovascular device, may have tremendous consequences. Accordingly, there is a need for replacing the mechanical control of endovascular devices with digital control mechanisms, thus providing precise control over the movements of endovascular devices within the body.

### SUMMARY

Embodiments consistent with the present disclosure provide systems, methods, and computer readable media generally relating to digital control of endovascular devices. The disclosed systems and methods may be implemented using a combination of conventional hardware and software as well as specialized hardware and software, such as a machine constructed and/or programmed specifically for performing functions associated with the disclosed method steps. Consistent with other disclosed embodiments, non-transitory computer readable storage media may store program instructions, which are executable by at least one processing device and perform any of the steps and/or methods described herein.

Consistent with disclosed embodiments, systems, methods, and computer readable media for digital control of an endovascular device are disclosed. The embodiments may include at least one processor. The at least one processor may be configured to obtain an input indicative of a first desired action of an endovascular device within a body structure of a patient. The at least one processor may also be configured to determine at least one property of a first force based on the input. The at least one processor may also be configured to cause, based on the determined at least one property, a control device of the endovascular device to exert the first force on a first portion of the endovascular device, the first portion of the endovascular device positioned outside the body of the patient. In disclosed embodiments, exertion of the first force may cause a second portion of the endovascular device to execute the first desired action within the body structure.

Consistent with disclosed embodiments, a control device for controlling movement of an endovascular device is disclosed. The control device may be configured to be positioned outside a body of a patient. The control device may include an input mechanism configured to receive input from a user; a device movement mechanism configured to control at least one movable portion of the endovascular device, the movable portion of the endovascular device configured for placement within the body of the patient; and at least one processor. The at least one processor may be configured to, in response to a first input, actuate the device movement mechanism to move the at least one movable portion of the endovascular device, so that the endovascular device is moved into a first configuration.

Consistent with disclosed embodiments, a control device for controlling an endovascular device is disclosed. The control device is configured to be positioned outside a body of a patient. The control device may include an input mechanism configured to receive an input from a user; a first mechanism for controlling a shaft of the endovascular device; and a second mechanism for controlling a core wire of the endovascular device. The control device may be configured to, in response to a first input from the user, actuate the first and second mechanisms to move the shaft of the endovascular device a first distance in a first direction and move the core wire of the endovascular device a second distance in a second direction that is opposite the first direction.

Consistent with disclosed embodiments, a control device for controlling an endovascular device is disclosed. The control device may be configured to be positioned outside a body of a patient. The control device may include an input mechanism configured to receive an input from a user; and a first mechanism for controlling a core wire of the endovascular device, the core wire extending through a shaft of the endovascular device. The control device may be configured to, in response to an input from the user, actuate the first mechanism to move the core wire of the endovascular device within the shaft of the endovascular device.

Consistent with disclosed embodiments, systems, methods, and computer readable media for endovascular treatment are disclosed. Embodiments may include an endovascular device configured for controllable movement at a treatment site within the body of a patient, the endovascular device including at least one movable portion. Embodiments may also include a control device for controlling the endovascular device. The control device may include an input mechanism configured to receive input from a user; a device movement mechanism configured to control the at least one movable portion of the endovascular device; and at least one processor. The at least one processor may be configured to, in response to a first input, actuate the device movement mechanism to move the at least one movable portion of the endovascular device, so that the endovascular device is moved into a first configuration at the treatment site.

Consistent with disclosed embodiments, systems, methods, and computer readable media for endovascular treatment are disclosed. Embodiments may include a control device for controlling an action of an endovascular device at a treatment site within the body of a patient. The control device may include a control device body configured to be positioned outside the body of the patient; a device movement mechanism configured to control at least one adjustable portion of the endovascular device, the at least one adjustable portion configured for placement at the treatment site within the body of the patient; and at least one magnet operably connected to the device movement mechanism. In response to a first actuation of the at least one magnet, the device movement mechanism may be configured to cause an action by the at least one adjustable portion of the endovascular device, so that the endovascular device transitions into a first configuration at the treatment site.

The forgoing summary provides certain examples of disclosed embodiments to provide a flavor for this disclosure and is not intended to summarize all aspects of the disclosed embodiments. Additional features and advantages of the disclosed embodiments will be set forth in part in the description that follows, and in part will be apparent from the description, or may be learned by practice of the disclosed embodiments. The features and advantages of the disclosed embodiments will be realized and attained by the elements and combinations particularly pointed out in the appended claims.

It is to be understood that both the foregoing general description and the following detailed description are examples and explanatory only and are not restrictive of the disclosed embodiments as claimed.

Unless otherwise defined, technical and/or scientific terms used herein have the same or similar meaning as commonly understood by one of ordinary skill in the art to which the present disclosure pertains. Examples of methods and/or materials are described below, but methods and/or materials similar or equivalent to those described may be used in the practice and/or testing of embodiments of the present disclosure. In cases of conflict, the patent specification, including definitions, will control. The materials, methods, and examples are illustrative only and are not intended to be necessarily limiting.

The accompanying drawings constitute a part of this specification. The drawings illustrate several embodiments of the present disclosure and, together with the description, serve to explain the principles of the disclosed embodiments as set forth in the accompanying claims.

### BRIEF DESCRIPTION OF THE DRAWINGS

The accompanying drawings, which are incorporated in and constitute a part of this specification, illustrate disclosed embodiments and, together with the description, serve to explain the disclosed embodiments. The particulars shown are by way of example and for purposes of illustrative discussion of embodiments of the present disclosure. The description taken with the drawings makes apparent to those skilled in the art how embodiments of the present disclosure may be practiced.

FIG. 1 depicts an example of an endovascular treatment system, consistent with disclosed embodiments.

FIG. 2 depicts a schematic view of a control device of an endovascular treatment system, consistent with disclosed embodiments.

FIG. 3A depicts an outer view of a first example of a control device of an endovascular treatment system, consistent with disclosed embodiments.

FIG. 3B depicts an outer view of a second example of a control device of an endovascular treatment system, consistent with disclosed embodiments.

FIG. 4A depicts an endovascular device with an expandable mesh in a contracted state, consistent with disclosed embodiments.

FIG. 4B depicts an enlarged view of the expandable mesh of FIG. 4A positioned at a treatment site within the body of a patient, consistent with disclosed embodiments.

FIG. 4C depicts the endovascular device of FIG. 4A with the expandable mesh in an expanded state, consistent with disclosed embodiments.

FIG. 4D depicts an enlarged view of the expandable mesh of FIG. 4C positioned at a treatment site within the body of a patient, consistent with disclosed embodiments.

FIG. 5A depicts an endovascular device with a guide wire in a straightened state, consistent with disclosed embodiments.

FIG. 5B depicts the endovascular device of FIG. 5A with the guide wire in a bent state, consistent with disclosed embodiments.

FIG. 6 depicts an endovascular device with a deflectable catheter, consistent with disclosed embodiments.

FIG. 7 is a flow chart illustrating an example of digitally controlling an endovascular device, consistent with some disclosed embodiments.

FIG. 8 depicts a graph showing changes over time in the size of an expandable mesh of an endovascular device, during a partially automated treatment of a hollow body organ with the endovascular device, consistent with disclosed embodiments.

FIGS. 9A-9C illustrate examples of a graphical user interface (GUI) of the endovascular treatment system of FIG. 1, consistent with disclosed embodiments.

FIG. 10 illustrates an example of a device movement mechanism of the control device of FIG. 2, consistent with disclosed embodiments.

FIG. 11 illustrates an example of a device movement mechanism of the control device of FIG. 2, consistent with disclosed embodiments.

FIG. 12 illustrates an example of a device movement mechanism of the control device of FIG. 2, consistent with disclosed embodiments.

FIG. 13 illustrates an example of a device movement mechanism of the control device of FIG. 2, consistent with disclosed embodiments.

FIG. 14 depicts an example of an endovascular treatment system, consistent with disclosed embodiments.

FIGS. 15A-15C illustrate examples of a magnet-controlled device movement mechanism of the endovascular treatment system of FIG. 14, consistent with disclosed embodiments.

FIG. 16 illustrates an example of a device movement mechanism of the endovascular treatment system of FIG. 14, consistent with disclosed embodiments.

FIG. 17 illustrates an example of a device movement mechanism of the endovascular treatment system of FIG. 14 consistent disclosed embodiments.

### DETAILED DESCRIPTION

Examples of embodiments are described with reference to the accompanying drawings. In the figures, which are not necessarily drawn to scale, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items or meant to be limited to only the listed item or items. It should also be noted that as used in the present disclosure and in the appended claims, the singular forms “a,” “an,” and “the” include plural references unless the context clearly dictates otherwise.

Unless specifically stated otherwise, as apparent from the following description, throughout the specification discussions utilizing terms such as “processing,” “calculating,” “computing,” “determining,” “generating,” “setting,” “configuring,” “selecting,” “defining,” “applying,” “obtaining,” “monitoring,” “providing,” “identifying,” “segmenting,” “classifying,” “analyzing,” “associating,” “extracting,” “storing,” “receiving,” “transmitting,” or the like, include actions and/or processes of a computer that manipulate and/or transform data into other data, the data represented as physical quantities, for example such as electronic quantities, and/or the data representing physical objects. The terms “computer,” “processor,” “controller,” “processing unit,” “computing unit,” and “processing module” should be expansively construed to cover any kind of electronic device, component or unit with data processing capabilities, including, by way of non-limiting example, a personal computer, a wearable computer, smart glasses, a tablet, a smartphone, a server, a computing system, a cloud computing platform, a communication device, a processor (for example, digital signal processor (DSP), an image signal processor (ISR), a microcontroller, a field programmable gate array (FPGA), an application specific integrated circuit (ASIC), a central processing unit (CPA), a graphics processing unit (GPU), a visual processing unit (VPU), and so on), possibly with embedded memory, a single core processor, a multi core processor, a core within a processor, any other electronic computing device, or any combination of the above.

The operations in accordance with the teachings herein may be performed by a computer specially constructed or programmed to perform the described functions.

As used herein, the phrase “for example,” “such as,” “for instance” and variants thereof describe non-limiting embodiments of the presently disclosed subject matter. Reference in the specification to features of “embodiments,” “one case,” “some cases,” “other cases” or variants thereof means that a particular feature, structure or characteristic described may be included in at least one embodiment of the presently disclosed subject matter. Thus, the appearance of such terms does not necessarily refer to the same embodiment(s). As used herein, the term “and/or” includes any and all combinations of one or more of the associated listed items. As used herein, the expression “at least one of . . . or” may include each listed item individually or any combination of the listed items. For example, the expression “at least one of A, B, or C” may include any of A, B, or C alone or any combination of A, B, and C (e.g., A+B, A+C, B+C, or A+B+C).

Features of the presently disclosed subject matter, are, for brevity, described in the context of particular embodiments. However, it is to be understood that features described in connection with one embodiment are also applicable to other embodiments. Likewise, features described in the context of a specific combination may be considered separate embodiments, either alone or in a context other than the specific combination.

In embodiments of the presently disclosed subject matter, one or more stages illustrated in the figures may be executed in a different order and/or one or more groups of stages may be executed simultaneously and vice versa. The figures illustrate a general schematic of the system architecture in accordance embodiments of the presently disclosed subject matter. Each module in the figures can be made up of any combination of software, hardware and/or firmware that performs the functions as defined and explained herein. The modules in the figures may be centralized in one location or dispersed over more than one location.

Examples of the presently disclosed subject matter are not limited in application to the details of construction and the arrangement of the components set forth in the following description or illustrated in the drawings. The subject matter may be practiced or carried out in various ways. Also, it is to be understood that the phraseology and terminology employed herein is for the purpose of description and should not be regarded as limiting.

In this document, an element of a drawing that is not described within the scope of the drawing and is labeled with a numeral that has been described in a previous drawing may have the same use and description as in the previous drawings.

The drawings in this document may not be to any scale. Different figures may use different scales and different scales can be used even within the same drawing, for example different scales for different views of the same object or different scales for the two adjacent objects.

Consistent with disclosed embodiments, “at least one processor” may constitute any physical device or group of devices having electric circuitry that performs a logic operation on an input or inputs. For example, the at least one processor may include one or more integrated circuits (IC), including application-specific integrated circuit (ASIC), microchips, microcontrollers, microprocessors, all or part of a central processing unit (CPU), graphics processing unit (GPU), digital signal processor (DSP), field-programmable gate array (FPGA), server, virtual server, or other circuits suitable for executing instructions or performing logic operations. The instructions executed by at least one processor may, for example, be pre-loaded into a memory integrated with or embedded into the controller or may be stored in a separate memory. The memory may include a Random-Access Memory (RAM), a Read-Only Memory (ROM), a hard disk, an optical disk, a magnetic medium, a flash memory, other permanent, fixed, or volatile memory, or any other mechanism capable of storing instructions. In some embodiments, the at least one processor may include more than one processor. Each processor may have a similar construction, or the processors may be of differing constructions that are electrically connected or disconnected from each other. For example, the processors may be separate circuits or integrated in a single circuit. When more than one processor is used, the processors may be configured to operate independently or collaboratively. The processors may be coupled electrically, magnetically, optically, acoustically, mechanically or by other means that permit them to interact.

Disclosed embodiments may include and/or access a data structure. A data structure consistent with the present disclosure may include any collection of data values and relationships among them. The data may be stored linearly, horizontally, hierarchically, relationally, non-relationally, uni-dimensionally, multidimensionally, operationally, in an ordered manner, in an unordered manner, in an object-oriented manner, in a centralized manner, in a decentralized manner, in a distributed manner, in a custom manner, or in any manner enabling data access. By way of non-limiting examples, data structures may include an array, an associative array, a linked list, a binary tree, a balanced tree, a heap, a stack, a queue, a set, a hash table, a record, a tagged union, ER model, and a graph. For example, a data structure may include an XML database, an RDBMS database, an SQL database or NoSQL alternatives for data storage/search such as, for example, MongoDB, Redis, Couchbase, Datastax Enterprise Graph, Elastic Search, Splunk, Solr, Cassandra, Amazon DynamoDB, Scylla, HBase, and Neo4J. A data structure may be a component of the disclosed system or a remote computing component (e.g., a cloud-based data structure). Data in the data structure may be stored in contiguous or non-contiguous memory. Moreover, a data structure, as used herein, does not require information to be co-located. It may be distributed across multiple servers, for example, that may be owned or operated by the same or different entities. Thus, the term “data structure” as used herein in the singular is inclusive of plural data structures.

Embodiments of the present disclosure relate to systems for performing various operations or functions described herein, including digital control of at least one endovascular device. Disclosed systems may be specially constructed for a particular purpose and/or may include at least one general-purpose processor selectively activated or configured by a software program executed by the at least one processor. In some embodiments, operations and functions performed by a disclosed system, or by at least one processor of a disclosed system, may additionally or alternatively be implemented as steps of a method or process or as operations performed when instructions contained in a non-transitory computer readable medium are executed (e.g., by at least one processor).

Embodiments of the present disclosure relate to methods for performing various operations or functions described herein, including digital control of at least one endovascular device. Aspects of methods disclosed herein may be implemented electronically, such as by at least one processor, and may occur over a network that is wired, wireless, or both wired and wireless. Aspects of methods disclosed herein may additionally, or alternatively, be implemented using non-electronic means. In a broadest sense, disclosed methods are not limited to particular physical and/or electronic instrumentalities (except where specified in the present disclosure or in the claims presented herein), but rather may be accomplished using many differing instrumentalities. In some embodiments, the steps of methods disclosed herein may be performed by features of disclosed systems (e.g., by at least one processor of a system disclosed herein) or may be implemented as operations performed when instructions contained in a non-transitory computer readable medium are executed (e.g., by at least one processor).

Embodiments of the present disclosure relate to non-transitory computer readable media containing instructions for performing various operations or functions described herein, including digital control of at least one endovascular device. Consistent with disclosed embodiments, non-transitory computer readable media may store program instructions executable by at least one processor and which, when executed, may cause the at least one processor to perform the steps and/or methods described herein. As used herein, a non-transitory computer readable medium may refer to any type of physical memory on which information or data readable by at least one processor can be stored. Examples may include random access memory (RAM), read-only memory (ROM), volatile memory, non-volatile memory, hard drives, CD ROMs, DVDs, flash drives, disks, and any other known physical storage medium. Singular terms, such as “memory” and “computer readable medium,” may, in some embodiments, refer to multiple structures, such as a plurality of memories or computer readable media. A computer readable medium may store instructions for execution by at least one processor, including instructions for causing the processor to perform steps or stages consistent with an embodiment disclosed herein. Additionally, one or more computer readable media may be utilized in implementing a computer-implemented method. The term “computer readable medium” should be understood to include tangible items and exclude carrier waves and transient signals.

Embodiments of the present disclosure relate to endovascular devices and to systems, methods, and non-transitory computer readable media for controlling endovascular devices. As used herein, an endovascular device may refer to any device or instrument configured to be placed within or to operate inside a blood vessel or another body structure or organ within a human body for a medical purpose, for example to diagnose and/or treat a patient. In disclosed embodiments, the body structure may include a hollow anatomical feature within the body of the patient. Non-limiting examples of a body structure include a blood vessel, capillary, aneurysm, esophagus, stomach, intestines, gallbladder, urethra, fallopian tubes, vaginal canal, urinary bladder, or any other hollow body structure or organ. In some embodiments, an endovascular device may include any device or instrument configured to be used during, or to otherwise facilitate, endovascular surgeries and procedures, as described in greater detail herein. An endovascular device may be configured to deliver a device, drug, or material from a first location (e.g., a location outside the body) to a treatment site in a blood vessel or other body structure and/or to remove a device, object, or material (e.g., a blood clot or other obstruction) from a blood vessel or other body structure. Additionally, or alternatively, in some embodiments, an endovascular device may be configured to cause a temporary or permanent change or transformation at a treatment site in a blood vessel (e.g., dilating a narrowed blood vessel or ablating vascular tissue). Some non-limiting examples of endovascular devices consistent with the present disclosure may include catheters (e.g., aspiration catheters or guide catheters), microcatheters, balloon catheters, devices with an expandable mesh (e.g., devices with an adjustable mesh and/or stent retrievers), medical sheaths, guide wires (e.g., controllable guide wires), coils, endovascular revascularization devices, embolization devices, ablation devices, stents, stent retrievers, or any other device configured to be placed within a blood vessel or other body structure.

Embodiments of the present disclosure relate generally to medical devices, methods and systems for treating occlusions in a body. More particularly, embodiments of the present disclosure relate to devices and methods for removing clots, including, but not limited to, emboli and thrombi from hollow body structures or organs, such as blood vessels. Additionally, or alternatively, embodiments of the present disclosure may also be utilized to dilate occluded hollow body structures or organs (e.g., blood vessels), as well as in other medical procedures where support of hollow body structures or organs (e.g., blood vessels) is desired. Examples of medical procedures include, but are not limited to, thrombectomy, vessel remodeling, vessel support, vessel dilation, angioplasty, and embolization of aneurysms. Furthermore, embodiments of the present disclosure may also be utilized to address other ailments to the vasculature of a body.

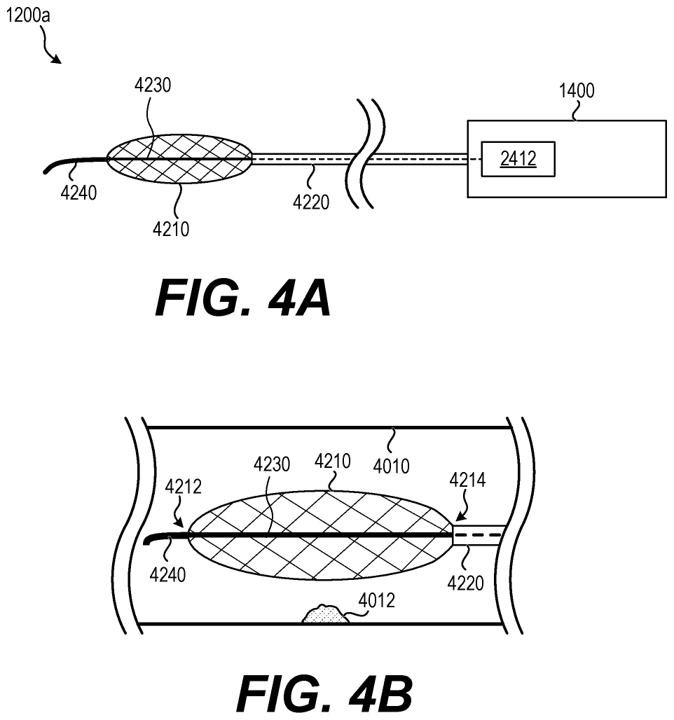

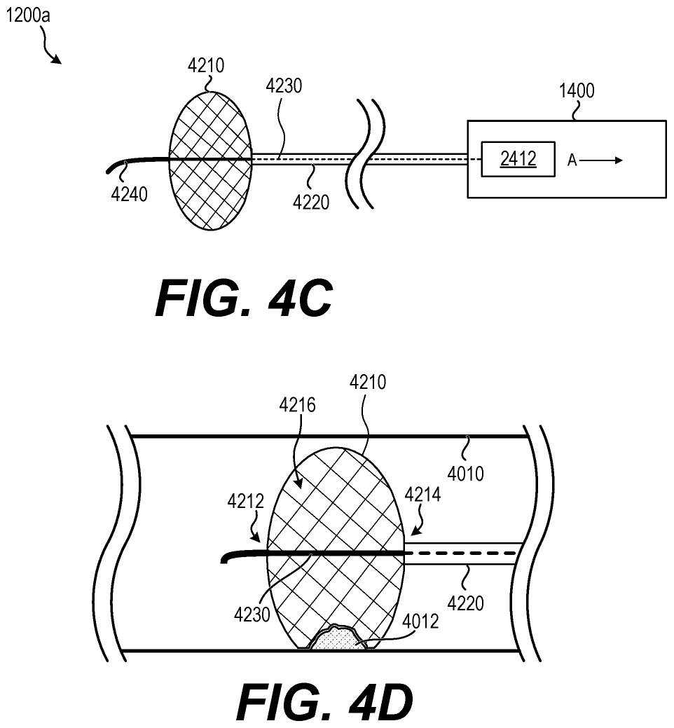

FIGS. 4A-4D depict a first non-limiting example of an endovascular device 1200a configured to be controlled with a control device 1400 (discussed in further detail below). Endovascular device 1200a may include a hollow, elongated shaft 4220 secured relative to control device 1400, as well as at least one expandable mesh segment 4210 connected to, or otherwise situated distal to, a distal end of the shaft 4220. Optionally, a flexible atraumatic tip 4240 may be secured to the distal end of the endovascular device 1200a. Mesh segment 4210 may be formed by a plurality of wires that are woven or coiled to form the expandable mesh. Endovascular device 1200a may also include a core wire 4230 (e.g., a control wire), which may be operated to cause radial expansion and contraction of mesh segment 4210 between a contracted state (FIGS. 4A-4B) and an expanded state (FIGS. 4C-4D). In the example shown, a first end of core wire 4230 may be secured to the distal end 4212 of the mesh segment (or to any other suitable portion of mesh segment 4210 or of endovascular device 1200a). Core wire 4230 may extend in one piece through the elongated shaft 4220 such that a second end of the core wire may be connected to control device 1400. For example, the second end of core wire 4230 may be operably connected to a device movement mechanism 2412 of the control device 1400. Device movement mechanism 2412 may be configured to axially move core wire 4230 in a proximal direction (to the right in FIGS. 4A-4D) or in a distal direction (to the left in FIGS. 4A-4D), thus moving the mesh distal end 4212 relative to the mesh proximal end 4214 and causing the mesh segment 4210 to expand or contract. Consistent with disclosed embodiments, endovascular device 1200a may be configured as a clot retrieval device, a device for mechanical dilation of body structures (e.g., a blood vessel), or a vascular remodeling device. Additionally, or alternatively, endovascular device 1200a may be configured to support a blood vessel during treatment of an aneurysm (e.g., while coils or other materials are delivered into the aneurysm to pack the aneurysm).

Mesh segment 4210 may have a reduced outer diameter (e.g., between 0.5 mm and 2.0 mm) in the contracted state, such that the mesh segment 4210 may be sized to be held within a delivery device. To expand the mesh, the distal end 4212 and proximal end 4214 of the mesh may be drawn closer together (e.g., by causing core wire 4230 to pull distal end 4212 in a proximal direction), causing the middle of mesh segment 4210 to expand outward toward vessel wall 4010. Thus, the outer diameter of mesh segment 4210 becomes larger when in the expanded state. In some embodiments, different degrees of expansion may be achieved with mesh segment 4210 by adjusting the distance between distal end 4212 and proximal end 4214. For example, mesh segment 4210 may be partially-expanded by moving distal end 4212 and proximal end 4214 of the mesh to be a first distance apart. In comparison, mesh segment 4210 may be fully-expanded by continuing to reduce the distance between the distal end 4212 and proximal end 4214 until a desired degree or size of expansion is achieved (e.g., until mesh segment 4210 contacts the vessel wall 4010).

In some embodiments, the wires forming mesh segment 4210 may be braided in a specific pattern for performing at least one action within a blood vessel. For example, the wires of mesh segment 4210 may be braided to form a clot capture mechanism, with openings 4216 (see FIG. 4D) between individual wires being sized to capture and retain occlusive material (e.g., blood clots) when the mesh segment is expanded. Additionally, or alternatively, the wires of mesh segment 4210 may be woven to form a clot anchoring segment configured to radially expand outward toward vessel wall 4010 to trap an obstruction 4012 (see FIG. 4D); when the expanded mesh segment 4210 is pulled proximally, it may push the trapped obstruction 4012 upstream so that the obstruction may be removed from the body.

Although the example shown in FIGS. 4A-4D includes a single mesh segment 4210, alternative embodiments of endovascular device 1200a may include two mesh segments, three mesh segments, four mesh segments, or any other suitable number of mesh segments. In embodiments including multiple mesh segments, each mesh segment may be individually expandable (e.g., each segment may include its own core wire) or, alternatively, all of the mesh segments may be configured to be expanded simultaneously (e.g., by a single core wire connected to the distal-most mesh segment).

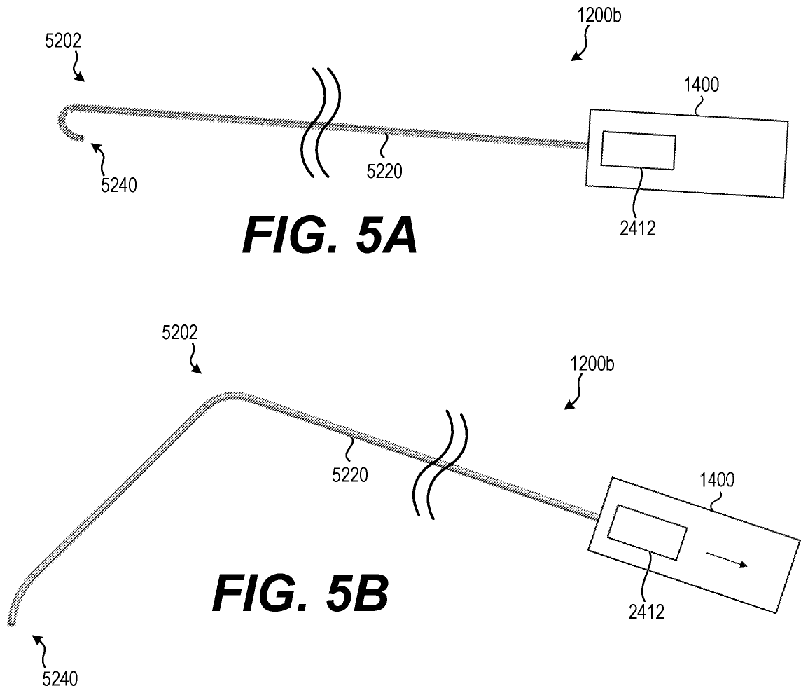

FIGS. 5A and 5B depict another non-limiting example of an endovascular device 1200b, which may be controlled with a control device 1400. Endovascular device 1200b may include a deflectable guide wire, including a hollow, elongated shaft 5220 (e.g., hypotube) connected to control device 1400, which may be configured to control bending and straightening of at least one bendable section 5202 of the guide wire. In some embodiments, elongated shaft 5220 (e.g., hypotube) may be sized and configured to traverse the human vasculature. In some embodiments, endovascular device 1200b may include an elongated coil secured relative to the distal end of elongated shaft 5220 (e.g., hypotube), as shown and described in U.S. Pat. No. 11,389,172, which is incorporated herein by reference. Endovascular device 1200b may additionally include a core wire (e.g., a control wire, not shown) that is connected to control device 1400 and which extends through elongated shaft 5220 (e.g., hypotube) to a point of connection with elongated shaft 5220 (e.g., at or near the distal portion of bendable section 5202 of endovascular device 1200b, such as the distal end 5420 of endovascular device 1200b). In some embodiments, the core wire of endovascular device 1200b may be operably connected to a device movement mechanism 2412 of control device 1400, which may be configured to axially move the core wire in a proximal direction (to the right in FIGS. 5A-5B) or in a distal direction (to the left in FIGS. 5A-5B). For example, to cause bending of the guide wire, device movement mechanism 2412 may pull the core wire in a proximal direction, which may pull the point of connection (e.g., distal end 5240) towards control device 1400 and cause bendable section 5202 to bend. To straighten the guide wire, for example, device movement mechanism 2412 may push the core wire in a distal direction, which may push the point of connection (e.g., distal end 5240) away from control device 1400 and cause bendable section 5202 to straighten (i.e., to reduce the degree of bending of bendable section 5202).

The example shown in FIGS. 5A-5B includes a single bendable section 5202. Alternative embodiments of endovascular device 1200b may include two bendable sections, three bendable sections, four bendable sections, or any other suitable number of bendable sections. In embodiments including multiple bendable sections, each bendable section may be individually bendable (e.g., each segment may include its own control wire).

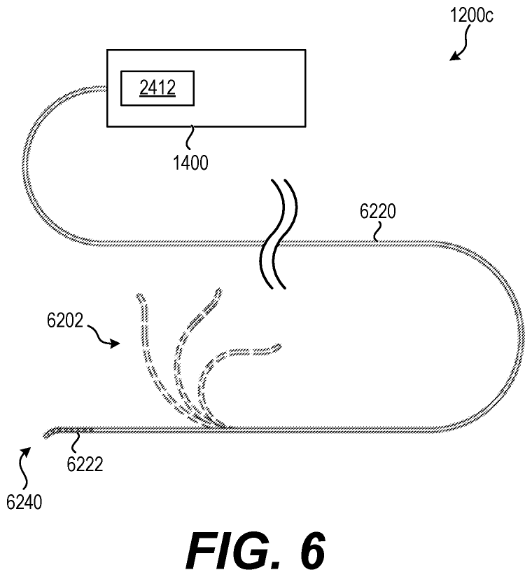

FIG. 6 depicts another non-limiting example of an endovascular device 1200c, which includes a deflectable catheter that may be controllably bent and straightened with control device 1400. The catheter of FIG. 6 may include an aspiration catheter, a guide catheter, or any other suitable type of catheter or medical sheath device. In some embodiments, the catheter of endovascular device 1200c may be configured for unidirectional bending, for bi-directional bending, or may have some other configuration for bending. Endovascular device 1200c may include a flexible, elongated sheath 6220 and optionally, a flexible coil segment 6222 arranged near the distal end of the endovascular device 1200c. Endovascular device 1200c may also include a core wire (e.g., a control wire, not shown) extending through elongated sheath 6220 and, in some embodiments, through coil segment 6222. A first end of the core wire may be secured to control device 1400, and a second end of the core wire may be secured relative to a deflectable segment 6202 of the catheter, such as a point of connection at or near the distal tip 6240 of the endovascular device.

In some embodiments, device movement mechanism 2412 may be secured to the proximal end of the core wire and may be configured to move the core wire axially to cause bending and straightening of the deflectable segment 6202 of endovascular device 1200c. As an example, in one implementation, device movement mechanism 2412 may push the core wire in a distal direction, causing the deflectable segment 6202 to curve in a first direction (e.g., clockwise in FIG. 6). Device movement mechanism 2412 may also pull the core wire in a proximal direction, causing the deflectable segment 6202 to curve in the opposite direction (e.g., counterclockwise in FIG. 6).

In some embodiments, an endovascular device may be a wire and/or a coil that may be controllably heated up and/or cut. The wire and/or coil may be used in an embolization procedure to treat aneurysm, or any other suitable endovascular procedure that may use a wire and/or coil. The wire may be flexible such that it is configured to coil inside an aneurysm, preventing blood from flowing into the aneurysm or from the aneurysm.

In some embodiments, device movement mechanism 2412 may be secured to the proximal end of the wire and may be configured to move the wire axially. As an example, in one implementation, device movement mechanism 2412 may push the wire in a distal direction, causing the wire to coil inside an aneurysm. Device movement mechanism 2412 may also pull the wire in a proximal direction.

Disclosed embodiments include systems, methods, and non-transitory computer readable media for digital control of an endovascular device or a plurality of endovascular devices. In some embodiments, a non-transitory computer readable medium is provided containing instructions that when executed by at least one processor cause the at least one processor to perform operations for digital control of an endovascular device. As used herein, a system for digital control of an endovascular device may refer to a system that uses at least one digital processor to control the actions of an endovascular device both inside and outside the body of a patient. Thus, in some embodiments, the endovascular device may be controlled and operated by the digital processor to perform an endovascular procedure, without a physician or other user manually operating the endovascular device. The user may be any medical staff member, such as but not limited to, an interventional radiologist, an interventional cardiologist, an interventional neurologist, a surgeon, a nurse and a technician. Similarly, a method for digital control of an endovascular device may refer to a method for operating or controlling the actions of an endovascular device with a digital computing device. In some embodiments, disclosed systems may include at least one processor is electronically connected to components which cause movement of, or other actions by, the endovascular device (e.g., a motor for causing movement of the endovascular device, or a mechanism for advancing or retracting a core wire of the endovascular device). The at least one processor may be configured to output a digital signal to the component, which may control the movement or other actions of the endovascular device based on the digital signal.

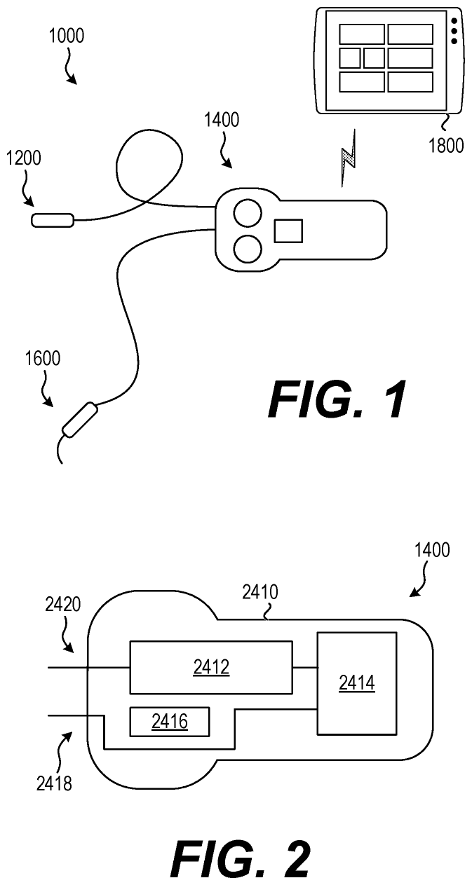

FIG. 1 depicts a non-limiting example of a system 1000 for digital control of an endovascular device 1200. Endovascular device 1200 may include any suitable endovascular device, including devices 1200a-c depicted in FIGS. 4A-6. System 1000 may include a control device 1400 configured to control the movements and other actions of endovascular device 1200. For example, and as discussed elsewhere in the present disclosure, control device 1400 may include an input mechanism for receiving input from a user and may be configured to control the endovascular device 1200 based upon the received user input. In some embodiments, control device 1200 may be configured to control more than one endovascular device 1200 (e.g., concomitantly or subsequent to one another).

In some embodiments, system 1000 may include at least one sensor 1600 configured to measure a characteristic of the body of the patient or of endovascular device 1200. For example, sensor 1600 may include a force sensor configured to measure a force exerted when endovascular device 1200 is removed from the blood vessel (e.g., when endovascular device 1200 is removed from the patient's body by control device 1400 or by a user). Sensor 1600 may provide sensor output to control device 1400, which may use the sensor output to control endovascular device 1200. FIG. 1 depicts a wired connection between control device 1400 and sensor 1600; in alternative embodiments, control device 1400 and sensor 1600 may be connected by a wireless connection (e.g., by Bluetooth®, Wi-Fi, or RF signals). In some embodiments, sensor 1600 may provide sensor output to a peripheral device 1800. Peripheral device 1800 and sensor 1600 may be connected by a wired connection. In other embodiments, peripheral device 1800 and sensor 1600 may be connected by a wireless connection (e.g., by Bluetooth®, Wi-Fi, or RF signals). According to other embodiments, sensor 1600 may provide sensor output to both control device 1400 and to peripheral device 1800. According to another embodiment, system 1000 may not include sensor 1600.

In some embodiments, system 1000 may include a peripheral device 1800 connected to control device 1400 by a wired and/or wireless connection. Peripheral device 1800 may include at least one processor and a user interface, such as a visual display or graphical user interface (GUI). For example, peripheral device 1800 may include a desktop computer, laptop computer, tablet, smartphone, surgical control device or panel, display screen, television, hand-held device, touchscreen device, or another appliance. In some embodiments, alerts and other feedback related to operation of endovascular device 1200 may be provided via the user interface of peripheral device 1800. Additionally, or alternatively, a physician or other user may provide input via the user interface of peripheral device 1800, which may relay the input to control device 1400 for controlling the actions of endovascular device 1200 based on the user's input.

FIG. 2 depicts a schematic view of control device 1400. In some embodiments, control device 1400 may be configured as a portable, handheld device, which a physician or other user may easily hold and manipulate during a procedure with endovascular device 1200. Control device 1400 may include a device cover 2410, a device movement mechanism 2412, a controller 2414, and a power source 2416 (e.g., a battery or an external power supply). In some embodiments, controller 2414 may include a printed circuit board (PCB) having a motor controller of device movement mechanism 2412, a data structure (e.g., a memory), and a communication mechanism (e.g., an antenna for wireless communication via radiofrequency signals or any other suitable communication medium). Control device 1400 may also include a sensor input 2418 for receiving output from sensor 1600. Accordingly, controller 2414 may be connected to a force sensor interface circuit for receiving output from sensor 1600. Control device 1400 may also include at least one endovascular device actuator 2420, which may provide a mechanical connection between endovascular device 1200 and device movement mechanism 2412. Actuator 2420 may be configured as a control feature of endovascular device 1200 (e.g., a control wire 4230 of endovascular device 1200a of FIGS. 4A-4D, a control wire of a guide wire, or a control wire of a guide catheter), such that movement of actuator 2420 may cause the endovascular device to perform a desired action. Device movement mechanism 2412 may be connected to actuator 2420 and may be configured to move actuator 2420 in an axial direction (e.g., to the left or right in FIG. 2), in a rotational direction, or any other desired movement, thereby controlling the movement and other actions of endovascular device 1200. In some embodiments, control device 1400 may include device movement mechanism 2412 only and no processor.

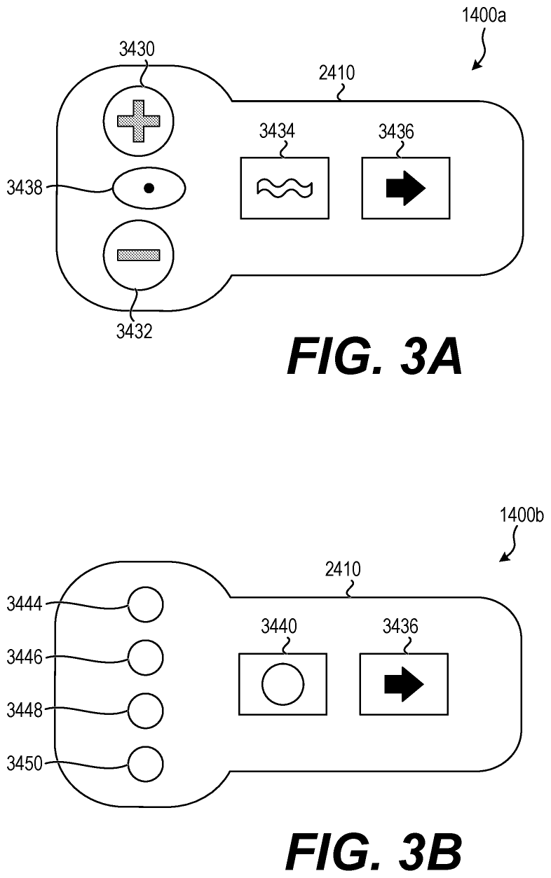

In disclosed embodiments, device movement mechanism 2412 may include at least one of a motor, an encoder, or a gear, which may be configured to move the at least one movable portion of endovascular device 1200. For example, controller 2414 may control the device movement mechanism 2412 to execute a desired movement of endovascular device 1200 using the at least one of the motor, encoder, or gear. In some embodiments, the at least one of the motor, encoder, or gear may be configured to move the at least one movable portion of the endovascular device 1400 based on at least one of a user input received by the input mechanism of control device 1400 (e.g., the control buttons shown in FIGS. 3A and 3B), a signal from at least one sensor (e.g., sensor 1600), a signal from a processor of a user interface device (e.g., peripheral device 1800), or computer-executable instructions for treatment with the endovascular device 1200, stored in a memory of controller 2414.

In some embodiments, control device 1400 may be configured to be operably connected to at least one peripheral device, such as device 1800. The peripheral device 1800 may include at least one of an image display screen, a control screen, or a computing device. The control device 1400 may be connected to the peripheral device 1800 by a wired connection and/or by a wireless connection, such as via Wi-Fi, Bluetooth®, or any other suitable communication medium.

Disclosed embodiments may include at least one processor, as defined elsewhere in the present disclosure. For example, disclosed systems for digital control of an endovascular device may include at least one processor. In FIG. 2, controller 2414 is an example of the at least one processor.

Disclosed embodiments may include obtaining an input indicative of a first desired action of an endovascular device within a body structure of a patient. For example, the at least one processor of the disclosed system may be configured to obtain an input indicative of a first desired action of an endovascular device within a body structure of a patient. As used herein, a first desired action of an endovascular device may include a movement of the endovascular device (e.g., a forward or distally-directed movement or a backwards or proximally-directed movement of the endovascular device), positioning of the endovascular device at a specified location (e.g., a treatment site within the body of a patient, or at a specified anatomical location such as at a branching of the body structure), removal of the endovascular device from the body of a patient, activation or deactivation of a component of the endovascular device (e.g., activation of an electrode of the endovascular device), a change or transformation of the endovascular device (e.g., an expansion or contraction of a mesh segment of the endovascular device, a bending in a distal portion of the endovascular device, or a temperature change), or any other desired outcome or effect to be achieved with the endovascular device.

In disclosed embodiments, the first desired action may include bending a distal portion of a guide wire (e.g., a distal tip of the guide wire) or a distal portion of a guide catheter within the body structure. Examples include bending at least one distal portion of the guide wire or guide catheter to a desired curvature and bending the at least one distal portion of the guide wire or guide catheter from a curved configuration into a straight configuration. As an illustration, FIGS. 5A-5B depict bending the bendable section 5202 of the guide wire of endovascular device 1200b from a straight configuration into a curved configuration at the distal end of the guide wire (FIG. 5A) or at another distal bendable portion of the device (FIG. 5B). As another example, FIG. 6 depicts bending a deflectable segment 6202 of the catheter of endovascular device 1200c.

Additionally, or alternatively, the first desired action may include an expansion or a contraction of a distal portion of the endovascular device. As used herein, expansion of a distal portion of the endovascular device may refer to an increase in the size or in at least one dimension of the endovascular device (e.g., an increase in an outer diameter, inner diameter, height, length or width of the distal portion of the endovascular device). Further, contraction may refer to a decrease in the size or in at least one dimension of the distal portion of the endovascular device, also referred to as collapse or relax. In some embodiments, the distal portion of the endovascular device may include a structure configured to expand and contract while the remainder of the device remains unchanged, such as an expandable mesh or stent or an inflatable balloon. For example, the first desired action may include expanding or contracting the distal portion of the endovascular device to achieve a specific size or degree of expansion or contraction, including a fully-contracted state, a fully-expanded state, or at least one intermediate state in between the expanded and contracted states. As an illustration, FIGS. 4A-4D depict expansion of an expandable mesh segment 4210 in a distal portion of endovascular device 1200a, from a contracted state (FIGS. 4A-4B) into an expanded state (FIGS. 4C-4D).

Additionally, or alternatively, the first desired action may include a movement of a tip of the endovascular device or of the entire endovascular device. For example, tip movement may include a forward (distally-directed) movement, a backward (proximally-directed) movement, a lateral or side-to-side movement, a vertical movement, bending, straightening, rotation, or any other desired movement of the tip of the endovascular device. Other examples include controlling advancement of the endovascular device to a desired location within the body of a patient or removing the endovascular device from the body of the patient. To illustrate, FIG. 6 depicts controlling a movement (specifically, bending) of a deflectable segment 6202 of endovascular device 1200c, including the distal tip 6240.

Additionally, in some embodiments, the first desired action may include a heating up or a cutting of an endovascular device. For example, the first desired action may include heating an endovascular device while the endovascular catheter is in the body of a patient to provide rapid and controlled rewarming in cases of hypothermia. As another example, the first desired action may include heating an endovascular device to cause a detachment of an endovascular coil during coil embolization. Further, in yet another example, the first desired action may include a mechanical detachment of an endovascular coil during coil embolization.

In some embodiments, the input may be obtained from a user interface and may constitute user input specifying the first desired action of the endovascular device. For example, the input indicative of the desired first action may include an input from a user performing a procedure with the endovascular device. Accordingly, the desired first action specified by the obtained input may correspond to the next step of the procedure being performed with the endovascular device. In some embodiments, the input from the user may be obtained from at least one of a manual input mechanism (e.g., a button, keyboard, computer mouse, lever, joystick, foot switch or pedal, or touch screen), an audio input mechanism (e.g., a microphone device configured to recognize verbal commands), a graphical user interface, or any other interface for receiving input from a user specifying the first desired action of the endovascular device.

In disclosed embodiments, the input from the user indicative of the first desired action may be obtained from a control handle operably connected to the at least one processor. For example, the user may operate an interface of control handle 1400 to indicate the first desired action to the at least one processor (e.g., to controller 2414). FIGS. 3A and 3B depict two non-limiting examples of a user interface of control device 1400. In the example depicted in FIG. 3A, control device 1400a may include an arrangement of buttons or other manual input mechanisms for specifying the first desired action of the endovascular device to the at least one processor. The user interface of control device 1400a may include a button 3430 for expanding an expandable portion of the endovascular device (e.g., a mesh segment or an inflatable balloon) and a button 3432 for contracting an expandable portion of the endovascular device (e.g., a mesh segment or an inflatable balloon). Additionally, button 3434 may cause activation or cessation of a pulsatile movement with an expandable portion of the endovascular device (e.g., of a mesh segment or an inflatable balloon); button 3436 may cause control device 1400a to move the endovascular device 1200 to a retrieval position (e.g., by contracting an expandable portion of the endovascular device 1200, e.g., the mesh segment or the inflatable balloon, and/or removing the endovascular device from the body structure and/or from the body of the patient); and button 3438 may cause an automatic operation for controlling the endovascular device 1200 with the control device 1400 (e.g., without further user input). For example, button 3438 may be pressed by a user to indicate that the device is in place and that automation may begin or resume. By operating the buttons in the user interface of control device 1400a, a user may specify the first desired action of the endovascular device 1200 to the at least one processor, which may control the endovascular device to execute the first desired action with a body structure of the patient.

FIG. 3B depicts another non-limiting example of an arrangement of buttons or other manual input mechanisms on the control device 1400b for specifying the first desired action of the endovascular device to the at least one processor. The user interface of control device 1400b may include a plurality of buttons 3444-3450 for indicating a desired size, shape, or dimension(s) of an expandable portion of the endovascular device 1200 and/or for indicating a size, shape, or dimension(s) of a body structure to be treated with the endovascular device 1200. For example, buttons 3444, 3446, 3448, and 3450 may signal to the control device 1400b to expand an expandable portion of the endovascular device 1200 (e.g., an expandable mesh or inflatable balloon) to have an outer diameter of 1 mm, 2 mm, 3 mm, and 4 mm, respectively. Although the illustrated example of control device 1400b shows four buttons 3444-3450 for specifying a size of the expandable portion of the endovascular device, more or fewer buttons may be implemented in alternative embodiments. Additionally, or alternatively, one or more buttons may be provided on control device 1400b for specifying specific values for other dimensions or characteristics of the endovascular device 1200, such as a degree of curvature or a length of the endovascular device advanced out from a delivery device into the body of the patient. A button 3440 may also be provided with control device 1400b for specifying automatic activation or cessation of a particular movement (e.g., a pulsatile movement or massage movement) with the endovascular device 1200, optionally for a predetermined period of time. A button 3436 (as discussed above) may also be provided with control device 1400b.

Persons of ordinary skill will understand that the buttons of control device 1400 depicted in FIGS. 3A and 3B are provided as a non-limiting example, and that any suitable configuration of buttons or other input structures, including buttons or other structures of different shapes, sizes, and/or colors, as well as any desired number of buttons or other structures, may be included in the user interface of control device 1400.

Additionally, or alternatively, the input from the user indicative of the first desired action may be obtained from a device including at least a second processor. The device including at least the second processor may include a device connected to the control device by a wired connection, wireless connection, or magnetic connection (further discussed in detail below) and optionally including a user interface for receiving input from the user indicating the first desired action of the endovascular device. Accordingly, the user input may be obtained remotely and transmitted to the control device from the device including at least the second processor. In disclosed embodiments, the user interface of the device including at least the second processor may include the features of the user interface of the control device, as discussed above. An example of a device including at least a second processor includes peripheral device 1800 of FIG. 1, as discussed elsewhere in the present disclosure.

In disclosed embodiments, the input indicative of the desired first action may additionally or alternatively include first data of at least one medical image captured prior to or during a procedure performed with the endovascular device. For example, the at least one medical image may be captured prior to the procedure showing a condition of a site in the patient's body to be treated during the procedure. Prior to an invasive procedure may refer to a time period before the invasive procedure is performed, before an endovascular device is inserted into the patient, and/or before the endovascular device reaches a certain location in the body of the patient. The data of the medical image may be used to plan the procedure with the endovascular device, including specific actions to be performed by the endovascular device at the treatment site within the body. Additionally, or alternatively, an imaging device may be provided during the procedure to capture at least one medical image of the treatment site and/or of the endovascular device. Non-limiting examples of the at least one medical image may include an angiogram, a computed tomography image, a magnetic resonance image, an ultrasound, or an X-ray. The data of the at least one medical image may be used as feedback to alter one or more parameters of the procedure (e.g., as feedback about the condition of the treatment site, information about the placement of the endovascular device at a specific anatomical location, or information about the condition of the endovascular device during the procedure) or to generate an alarm when an action by the endovascular device exceeds a safety threshold (e.g., when an obstruction is visualized in the patient's body that may damage the endovascular device, when the endovascular device comes in contact with the incorrect part of the patient's body, or when too much force is exerted by the endovascular device such as on the body structure). With reference to FIG. 1, the imaging device may be included in system 1000 as a peripheral device 1800. Thus, in some embodiments, the peripheral imaging device 1800 may capture the at least one medical image, process the at least one medical image, and transmit data of the at least one medical image to control device 1400 and/or to the at least one processor of system 1000. Additionally, or alternatively, the imaging device may be included in system 100 as an additional imaging device (not shown in FIG. 1). Thus, in some embodiments, the additional imaging device may capture the at least one medical image, process the at least one medical image, and transmit data of the at least one medical image to peripheral device 1800, to control device 1400, and/or to the at least one processor of system 1000. The transmitted data of the at least one medical image may include raw medical image data, processed medical image data, or data generated from analysis of the at least one medical image by the peripheral device 1800 and/or by the additional imaging device.

In disclosed embodiments, the input indicative of the desired first action may additionally or alternatively include second data derived from at least one sensor output. For example, a sensor may be provided with disclosed systems for monitoring the patient's body and/or a condition of the endovascular device. Output from the sensor may be used as feedback for controlling the actions of the endovascular device. In FIG. 1, sensor 1600 may be used to monitor the patient's body and/or the endovascular device and may provide sensor output to control device 1400. For example, sensor 1600 may be configured as a force meter for measuring a force exerted when endovascular device 1200 is removed from the body structure (e.g., blood vessel) by control device 1400 or by a user. When the force measured by sensor 1600 exceeds a threshold, an alarm may be generated to notify the user (e.g., via a user interface of control device 1400, or via peripheral device 1800, or via an element in sensor 1600, such as a vibration of sensor 1600) and/or an automated safety protocol may be executed by the control device 1400 to reduce the magnitude of the force exerted by the endovascular device or to halt the action. For example, an automated safety protocol for retrieval of an endovascular device from a body structure may close the expandable mesh segment or the inflatable balloon to reduce the magnitude of the force exerted by the endovascular device prior to continuation of the action.

Disclosed embodiments may include determining at least one property of a first force based on the input indicative of the first desired action. For example, disclosed systems may include at least one processor configured to determine at least one property of a first force based on the input indicative of the first desired action. In some embodiments, the “first force” may refer to a force exerted by control device 1400 on a first, proximal portion of the endovascular device 1200 in order to cause a second, distal portion of the endovascular device to execute the first desired action. The at least one property of the first force may include at least one of a magnitude of the first force, a time duration of the first force, or a direction of the first force.

For example, with reference to FIGS. 4A-4D, the obtained input may indicate a desired expansion size of expandable mesh segment 4210 (e.g., the input may indicate that mesh segment 4210 is to be expanded to have an outer diameter of 2 mm to 15 mm, such as but not limited to, 2 mm, 2.5 mm, 3 mm, 3.5 mm, 4 mm, 5 mm, 6 mm, 7 mm, 8 mm, 9 mm, 10 mm). In some embodiments, the at least one processor may determine the current size of mesh segment 4210, such as from imaging data (e.g., from a peripheral device 1800), sensor output data from a sensor 1600, and/or historical data of movement of the endovascular device 1200. Based on the current size of the mesh segment 4210, the at least one processor may determine both a direction of movement (e.g., expansion vs. contraction of a mesh segment) and a degree of movement (e.g., a change in the outer diameter of mesh segment 4210, or a change in the distance between distal end 4212 and proximal end 4214 of the mesh segment) to achieve the desired size of the mesh segment 4210. In addition, the at least one processor may determine or obtain a ratio between a movement of the mesh segment 4210 (e.g., a change in the outer diameter of mesh segment 4210, or a change in the distance between distal end 4212 and proximal end 4214 of the mesh segment) and the corresponding movement of the actuator 2420 by the device movement mechanism 2412. Based on this information, the at least one processor may determine properties of the first force, including a direction and a magnitude, to be exerted by the device movement mechanism 2412 on the actuator 2420 in order to adjust the mesh segment 4210 to have the indicated size (i.e., to execute the first desired action by endovascular device 1200 within the body structure).

In another example, with reference to FIGS. 5A-5B, the obtained input may indicate a desired bending curvature of a distal end 5240 of endovascular device 1200b (e.g., guide wire) to advance through tortuous anatomy. In some embodiments, the at least one processor may determine the current bending curvature of distal end 5240, such as from imaging data (e.g., from a peripheral device 1800), sensor output data from a sensor 1600, and/or historical data of movement of the endovascular device 1200. Based on the current bending curvature of distal end 5240, the at least one processor may determine both a direction of movement of a core wire (e.g., pulling vs. pushing of a core wire to bend or straighten distal end 5240, respectively) and a speed of movement of the core wire. In addition, the at least one processor may determine or obtain a ratio between a movement of the core wire and the corresponding movement of the actuator 2420 by the device movement mechanism 2412. Based on this information, the at least one processor may determine properties of the first force, including a direction and a magnitude, to be exerted by the device movement mechanism 2412 on the actuator 2420 to adjust the distal end 5240 to have the indicated bending curvature (i.e., to execute the first desired action by endovascular device 1200 within the body structure).

Disclosed embodiments may include causing a control device of the endovascular device to exert the first force on a first portion of the endovascular device based on the determined at least one property of the first force. For example, disclosed systems may include at least one processor configured to cause a control device of the endovascular device to exert the first force on a first portion of the endovascular device based on the determined at least one property of the first force. As used herein, the “first portion of the endovascular device” may include the part of endovascular device 1200 that interacts with, and is manipulated by, the control device 1400. An example of a first portion of the endovascular device includes actuator 2420 of FIG. 2, which may include a control wire or other steering mechanism that is manipulated by control device 1400 to achieve a desired action with the endovascular device 1200. For example, in FIGS. 4A-4D, the “first portion” of endovascular device 1200a may include the proximal end of control wire 4230 which is operably connected to device movement mechanism 2412. Similarly, in FIGS. 5A-5B and 6, the “first portion” of endovascular devices 1200b and 1200c may include the proximal ends of the control wire of each endovascular device, which may be manipulated by device movement mechanism 2412 to cause bending and straightening of bendable portions 5202 and 6202. In disclosed embodiments, the first portion of the endovascular device may be positioned outside the body of the patient. For example, the first portion of the endovascular device may be connected to, or at least located in proximity to, the control device 1400 which may also be positioned outside the patient's body during the procedure with endovascular device 1200.

In disclosed embodiments, exertion of the first force on the first portion of the endovascular device may cause a second portion of the endovascular device to execute the first desired action within the body structure. In some embodiments, a “second portion of the endovascular device” may include the part of the endovascular device 1200 that is located in or near the treatment site during the procedure with the endovascular device. This may include the distal portion or the distal end of the endovascular device 1200. In some embodiments, a “second portion of the endovascular device” may include the part of the endovascular device 1200 that is used to navigate through tortuous anatomy (e.g., distal bendable section). In disclosed embodiments, the second portion of the endovascular device may include the structure that is used to perform the desired treatment at the treatment site. For example, the second portion of the endovascular device may include at least one of a distal portion of a guide wire, a distal portion of a guide catheter, a distal portion of a catheter (e.g., distal portion of an aspiration catheter), an expandable distal portion of the endovascular device (e.g., mesh segment), or a distal inflatable section of the endovascular device (e.g., inflatable balloon). To illustrate, the second portion of endovascular device 1200 may include expandable mesh segment 4210 of FIGS. 4A-4D, bendable section 5202 of the guide wire of FIGS. 5A and 5B, and deflectable segment 6202 of the catheter of FIG. 6.

In disclosed embodiments, the input indicating the first desired action may be indicative of a navigation target. As used herein, a navigation target may refer to a target site within the body of the patient where a particular part of the endovascular device (e.g., the distal portion such as the distal end) is to be placed. In some embodiments, the navigation target may be indicated by coordinate data, by route data, or by other data. The at least one processor may be configured to determine the at least one property of the first force based on the navigation target. For example, the at least one processor may determine a current location of the endovascular device 1200 (e.g., from imaging data, such as from a peripheral device 1800, from sensor output from a sensor 1600, and/or from movement history data of the endovascular device 1200) and may calculate a distance and/or a navigation route for advancing the endovascular device 1200 to the navigation target. The at least one processor may determine properties of the first force, such as a magnitude and direction of the applied first force as a function of time, in order to steer the endovascular device 1200 through the patient's body to arrive at the navigation target.

In disclosed embodiments, the at least one property of the first force may be determined based on information about at least one of the endovascular device, the body structure to be treated, the location of the body structure to be treated, the indication to be treated (e.g., a narrowing in a body structure or an obstruction in a body structure), or other structures at or near the treatment site. For example, the at least one property of the first force may be determined based on a characteristic of the endovascular device or the position of the endovascular device in the body structure. The characteristic of the endovascular device may include information about the device's current size (e.g., diameter), configuration (e.g., expanded, contracted, bent, straightened, inflated, deflated), or location, which may be used as input to determine the force required to move the endovascular device from its current location and configuration to the desired location and configuration. In addition, information about the endovascular device's position in the body structure may indicate an additional movement required to move the endovascular device to a navigation target, may indicate that the endovascular device has migrated away from the desired treatment site and may come in contact with tissue that is not to be treated, or may indicate that the endovascular device has migrated away from the desired navigation route required to reach a navigational target. The characteristic of the endovascular device may also include information about individual components included in the endovascular device (e.g., one or more markers indicating one or more portions of the endovascular device), a material of the endovascular device, or a mechanism for activating or deactivating a function of the endovascular device.

Additionally, or alternatively, the at least one property of the first force may be determined based on a characteristic of the body structure, such as a size or shape of the body structure, a curvature or flatness of the body structure, a diameter of the body structure, a type of tissue forming the body structure, and a medical or surgical history of the body structure. Additionally, or alternatively, the at least one property of the first force may be determined based on a type of obstruction or a type of narrowing in the body structure. For example, a type of obstruction may include a blood clot, a plaque, or a foreign body. Additionally, a type of narrowing in the body structure may include a vasospasm or atherosclerosis (e.g. intracranial artery stenosis). Based on the type of obstruction or narrowing, the at least one processor may determine the magnitude of the first force (e.g., as some types of obstructions require more force to overcome), as well as a direction and timing of the first force for achieving a movement of the second portion of the endovascular device that is most suitable for the type of obstruction or narrowing.

In disclosed embodiments, the at least one processor may be configured to determine the at least one property of the first force for causing the second portion of the endovascular device to perform a movement at a substantially constant speed. For example, the at least one processor may determine the amount of force to be applied on the actuator 2420 to overcome any resistance at the treatment site (e.g., due to a blood clot or obstruction in contact with the second portion of the endovascular device) and to achieve the substantially constant speed, as well as the direction of the first force that corresponds to the direction of movement of the endovascular device. The at least one processor may also determine the duration of the application of the first force, so that the endovascular device is moved at the substantially constant speed for the desired length of time.

Additionally, or alternatively, the at least one processor may be configured to determine the at least one property of the first force for causing the second portion of the endovascular device (e.g., expandable mesh 4210) to expand or to contract. For example, the at least one property may include a direction of the first force to achieve the desired one of expansion or contraction of the second portion of the endovascular device 1200, such as to a predetermined diameter size. Additionally, or alternatively, the at least one processor may be configured to determine the at least one property of the first force for causing the second portion of the endovascular device to perform a repetitive motion (also referred to as a pulsatile movement). For example, in order to perform a repetitive massaging motion with expandable mesh 4210, the at least one processor may determine a corresponding repetitive first force to be exerted on the actuator 2420 to achieve a predetermined expansion and/or contraction diameter for maximal clot integration. The at least one property of the repetitive first force may include a duration of each cycle of the repetitive first force, a direction of moving the actuator 2420 as a function of time, and the magnitude of force to be exerted on the actuator 2420 to achieve the desired type, duration, and number of repetitions of the repetitive motion with the expandable mesh 4210. In some embodiments, the duration of each cycle of the repetitive first force may be 1 second to 3 minutes, such as but not limited to, 1 sec, 5 sec, 10 sec, 15 sec., 20 sec., 30 sec., 60 sec., 90 sec., 120 sec, 150 sec, 180 sec. Additionally, or alternatively, the at least one processor may be configured to determine the at least one property of the first force for causing the second portion of the endovascular device to bend or to straighten. For example, the at least one processor may determine the direction of the first force to achieve the desired one of bending or straightening the endovascular device, as well as a magnitude of force required for changing the curvature of the second portion of the endovascular device. Additionally, or alternatively, the at least one processor may be configured to determine the at least one property of the first force for causing the second portion of the endovascular device to inflate or to deflate.

In some embodiments, the at least one processor may utilize medical image data (such as image data obtained as input) for determining properties of the first force. For example, the at least one processor may be configured to calculate a convolution of the at least one medical image to derive a convolution value. Based on the derived convolution value, the at least one processor may be configured to determine the at least one property of the first force. For example, a convolution of at least part of the medical image may be calculated, and the digital signal may be generated based on a value of the calculated convolution.

Additionally, the at least one processor may use medical image data to determine feedback about the treatment site, feedback about the endovascular device, and//or feedback about the anatomical location of the endovascular device in the body structure. The determined feedback may be used to determine properties of the first force. For example, the at least one processor may be configured to detect, in the at least one medical image, at least one of the body structure or the second portion of the endovascular device in the body structure. Based on the detected information from the at least one medical image, the at least one processor may determine whether the second portion of the endovascular device is located in the correct position within the body structure and whether the endovascular device has the correct size or configuration. The at least one processor may be configured to determine the at least one property of the first force based on the detection of the at least one of the body structure or the second portion of the endovascular device in the body structure. For example, the at least one processor may determine from the image data whether an adjustment of the position, size, or configuration of the endovascular device is needed. The at least one processor may then determine the properties of the first force to achieve the desired adjustment.

In disclosed embodiments, the at least one processor may be configured to analyze the at least one medical image to determine a position of the second portion of the endovascular device with respect to a second object. For example, the second object may include a clot or other obstruction, a portion of another device (e.g., a second endovascular device), a site of vessel narrowing, or another anatomical structure. The at least one processor may be configured to determine the at least one property of the first force based on the determined position of the second portion of the endovascular device with respect to the second object. For example, the at least one processor may determine the at least one property of the first force for the second portion of the endovascular device to avoid the second object, if desired. Alternatively, the at least one processor may determine the at least one property of the first force for the second portion of the endovascular device to perform a desired interaction with the second object, such as by removing a detected clot or radially expanding a site of vessel narrowing.

As discussed above, the obtained input indicating the first desired action may include data derived from a sensor output. In disclosed embodiments, the sensor output may indicate a force exerted to remove the endovascular device from the body structure. For example, the sensor device 1600 may include a force meter configured to measure a magnitude of the force exerted by the endovascular device 1200 when retrieved from the body structure. Thus, for example, when an endovascular device 1200, such as a device with an expandable mesh 4210, is expanded to contact the inner wall 4010 of a blood vessel, force applied to retrieve endovascular device 1200 may damage the vessel wall. Additionally, or alternatively, force applied to retrieve endovascular device 1200 through tortuous anatomy may result in exerted force causing tissue damage. The at least one processor may receive output from the force meter 1600 and determine when a force being applied to remove a mesh 4210 from the blood vessel 4010 exceeds a predetermined safety threshold. In some embodiments, when the threshold is exceeded, the at least one processor may output an alert (e.g., via a user interface of control device 1400, via peripheral device 1800, or via an element in sensor 1600) and/or may change the operation of the endovascular device within the body (e.g., by reducing the application of force by the endovascular device or by halting the action).

In disclosed embodiments, the at least one processor may be configured to determine, based on the obtained input, that the first desired action of the endovascular device in the body structure exceeds a predetermined threshold. For example, the threshold may indicate a maximum application of force by the endovascular device on the body structure, or a minimum distance that must be maintained between the endovascular device and a tissue that is not to be treated with the endovascular device. In some embodiments, the at least one processor may determine that the threshold is exceeded based on output from sensor device 1600, such as a force meter discussed above. Additionally, or alternatively, the at least one processor may utilize image data to evaluate the respective locations of the endovascular device and the patient's anatomy at and around the treatment site. The at least one processor may determine that the threshold is exceeded when the endovascular device is determined to be in the incorrect location or at a distance that is too close to an anatomy that is not to be treated.

In disclosed embodiments, the at least one processor may be configured to output an alert based on the determination that the first desired action of the endovascular device in the body structure exceeds the threshold. For example, the at least one processor may output a graphical alert, an audio alert, and/or a sensory alert (e.g., vibration) to the user via a user interface of control device 1400, via a peripheral device 1800, or via an element in sensor 1600. Additionally, or alternatively, the at least one processor may be configured to alter at least one parameter of the first force based on the determination that the first desired action of the endovascular device in the body structure exceeds the threshold. For example, the at least one processor may cause the device movement mechanism 2412 to reduce the magnitude of force exerted on actuator 2420, which in turn reduces the force exerted by endovascular device 1200 on the body structure. Alternatively, the at least one processor may cause the device movement mechanism 2412 to stop all movement of the endovascular device, so that a dangerous application of force or movement of the endovascular device is stopped immediately.

In disclosed embodiments, it may be desired to execute a second desired action with the endovascular device after executing the first desired action. For example, each action may be considered a step in a surgical procedure, with the steps following each other in a sequence. Accordingly, the at least one processor may be configured to determine at least one property of a second force based on an obtained input indicating a second desired action of the endovascular device within the body structure. In some embodiments, the at least one property of the second force may be determined based on the same input as the first force, discussed above. Additionally, or alternatively, the at least one property of the second force may be determined based on a second input. Like the first input discussed above, the second input may include at least one of an input from a user performing a procedure with the endovascular device, first data of at least one medical image, or second data derived from at least one sensor output. In disclosed embodiments, the at least one processor may be configured to cause the control device of the endovascular device to exert the second force after causing the control device to exert the first force on the first portion of the endovascular device. Similar to exertion of the first force discussed above, the at least one processor may cause the control device of the endovascular device to exert the second force based on the determined at least one property of the second force. For example, in FIGS. 1-2, the at least one processor may cause control device 1400 to exert the second force on endovascular device 1200 based on the determined at least one property of the second force.

In some embodiments, the second desired action of endovascular device 1400 may be executed by exerting the second force on the first portion of the endovascular device (that is, on the same portion of endovascular device 1400 that was controlled to perform the first desired action). For example, control device 1400 may be configured to exert the first force and the second force on actuator 2420, which may exert the corresponding forces on endovascular device 1200. Alternatively, the control device may exert the second force on a third portion of the endovascular device that is different from the first portion of the endovascular device. For example, in some embodiments the control device 1400 may include multiple actuators 2420, each of which may connect to, and control movement of, different parts of the endovascular device 1200. In such embodiments, two separate actuators 2420 may correspond to the first portion and third portion of the endovascular device. Further, the two different parts of endovascular device 1200 that are respectively controlled by the different actuators may correspond to the second portion and a fourth portion of the endovascular device. In disclosed embodiments, exertion of the second force may cause at least one of the second portion of the endovascular device or a fourth portion of the endovascular device to execute a second desired action within the body structure. For example, in some embodiments, exertion of the first force and second force may cause the same part of the endovascular device 1200 to perform the first and second desired actions. For example, the first force and second force may be applied to the same actuator 2420 for causing movement of the same part of the endovascular device 1200. Alternatively, exertion of the first force and second force may cause two different parts of the endovascular device 1200 (specifically, the second portion and fourth portion, respectively) to perform the first and second desired actions. For example, endovascular device 1200 may include multiple actuators 2420 that control different parts of the device (e.g., which may cause bending of bendable segment 5202 of FIGS. 5A-5B in opposite directions, or which may cause bending of two separate bendable segments 5202 in the same endovascular device of FIGS. 5A-5B). The first and second forces may be exerted on different actuators 2420, causing different actions by the endovascular device (e.g., causing bendable segment 5202 to bend in different directions, in subsequent steps or concomitantly).

In disclosed embodiments, the second desired action may include at least one of completing the first desired action of the endovascular device or executing another desired action of the endovascular device that differs from the first desired action. In disclosed embodiments, the second desired action may include a contraction of an expanded distal portion of the endovascular device. For example, the first desired action may include expanding mesh segment 4210, while the second desired action may include contracting the expanded mesh segment 4210. In another example, the first desired action may include expanding mesh segment 4210 to one diameter, while the second desired action may include expanding the mesh segment 4210 to a second diameter (e.g., to a fully expanded diameter). In yet another example, the first desired action may include contracting mesh segment 4210 to one diameter, while the second desired action may include contracting the mesh segment 4210 to a second diameter (e.g., to a fully collapsed diameter). Additionally, or alternatively, the second desired action may include removing the second portion of the endovascular device from the body structure. For example, a user may indicate the second direction action by pressing button 3436 of FIG. 3A or 3B, which may cause an expanded mesh segment 4210 to be contracted to its fully-contracted state and removed from the body of the patient.

In some embodiments, the at least one property of the second force may be determined before exerting the first force on the first portion of the endovascular device, such as in a sequence of steps in a predetermined surgical plan. Additionally, or alternatively, the at least one property of the second force may be determined based on a second input from a user performing a procedure using the endovascular device. For example, the user may provide the second input using one of the buttons of control handles 1400a or 1400b of FIGS. 3A-3B. Additionally, or alternatively, the second input may be received after causing the control device to exert the first force on the first portion of the endovascular device. For example, the second input may be received from a user, as discussed above, as data from a sensor 1600, as data from an imaging device 1800, and/or as data from an additional imaging device (not shown) that provides feedback about the execution of the first desired action by the endovascular device. For example, the at least one property of the second force may be determined based on an analysis of at least one medical image captured (e.g., by a peripheral imaging device 1800 or by an additional imaging device) after causing the control device to exert the first force on the first portion of the endovascular device.

Similar to determining the at least one property of the first force discussed above, the at least one processor may be configured to determine the at least one property of the second force for causing the second portion of the endovascular device to perform a movement (e.g., at a substantially constant speed), for causing the second portion of the endovascular device to expand, for causing the second portion of the device to inflate, for causing the second portion of the device to deflate, for causing the second portion of the endovascular device to contract, for causing the second portion of the endovascular device to perform a repetitive motion, for causing the second portion of the endovascular device to bend, and/or for causing the second portion of the endovascular device to straighten.

Returning to FIGS. 1-3B, embodiments related to control device 1400 are described herein. In some embodiments, a control device for controlling movement of an endovascular device may be provided. The control device may be configured to be positioned outside a body of a patient. For example, control device 1400 may be configured to control movement of endovascular device 1200a of FIGS. 4A-4D by moving control wire 4230 proximally and/or distally, which may cause expansion and contraction of mesh segment 4210. Similarly, control device 1400 may be configured to control movement of endovascular devices 1200b and 1200c by moving their respective control wires, thus causing bending and/or straightening of deflectable segment 5202 and deflectable segment 6202. In some embodiments, the control device may include an input mechanism configured to receive input from a user. Examples of an input mechanism include the arrangement of at least one input structure (e.g., buttons) depicted in FIGS. 3A and 3B. Additionally, or alternatively, the input mechanism of control device 1400 may include at least one of a button, a keyboard, a computer mouse, a lever, a joystick, or a touch screen.

In disclosed embodiments, the control device may include a device movement mechanism configured to control at least one movable portion of the endovascular device. For example, and as discussed above, device movement mechanism 2412 may be configured to control expansion and contraction of an expandable mesh 4210, bending and straightening of a deflectable segment 5202 of a guide wire, and bending and straightening of a deflectable segment 6202 of a catheter. The movable portion of the endovascular device may be configured for placement within the body of the patient, including while the movable portion is moved under control of the control device. In disclosed embodiments, the control device may also include at least one processor, an example of which may include controller 2414 shown in FIG. 2.

In some embodiments, the at least one processor of the control device (e.g., controller 2414) may be configured to actuate the device movement mechanism in response to a first input. The first input may be received from a user via the input mechanism of the control device (e.g., one of the buttons depicted in FIGS. 3A and 3B). Additionally, or alternatively, the first input may be received from a user via a user interface of peripheral device 1800 (examples of which may include a touch screen or GUI). For example, a physician may digitally control a procedure with endovascular device 1200 using peripheral device 1800, which may provide a larger user interface than control device 1400 in some embodiments. Additionally, or alternatively, the first input may be obtained from a sensor device 1600 (as discussed above). Additionally, or alternatively, the first input may correspond to computer-executable instructions for treatment with the endovascular device 1200, stored in a memory (e.g., a memory component of controller 2414).

In some embodiments, the at least one processor of the control device (e.g., controller 2414) may be configured to, in response to the first input, actuate the device movement mechanism to move the at least one movable portion of the endovascular device so that the endovascular device is moved into a first configuration. For example, the first input may indicate a desired action or movement of the endovascular device (e.g., a user may push one of buttons 3430 or 3432 shown in FIG. 3A, which may indicate an instruction to the control device 1400 to expand or contract an expandable mesh or to bend or straighten a guide wire or catheter). In response, the device movement mechanism 2412 may execute the requested movement of the endovascular device 1200 (e.g., by manipulating a control wire or other actuator of the endovascular device). As another example, the first input may indicate a desired configuration of the endovascular device (e.g., a user may push one of the buttons 3444-3450 of FIG. 3B, which may indicate a certain arrangement of, or action by, the endovascular device (e.g., a desired expansion diameter or shape of an expandable mesh 4210, such as a fully-expanded state, a fully-contracted state, a state of 90% expansion, a state of 80% expansion, etc., a desired expansion time (e.g., an expansion to occur over a time period ranging between 10 seconds and 3 minutes), or a desired contraction time (e.g., a contraction to occur over a time period ranging between 10 seconds and 3 minutes)) or, alternatively, which may indicate a characteristic of a treatment site in the patient's body to be treated with the endovascular device 1200 (e.g., a diameter of a body structure to be treated with the endovascular device, a type of narrowing or obstruction to be treated with the endovascular device, such as a blood clot, vasospasm, plaques, or a type of clot to be treated with the endovascular device, such as blood clots and clots comprising fatty or calcium deposits)). In response, the device movement mechanism may control the endovascular device actuator 2420 (e.g., a control wire) to move the endovascular device 1200 into the shape or configuration corresponding to the received input.

In disclosed embodiments, the at least one processor of the control device (e.g., controller 2414) may be configured to actuate the device movement mechanism in response to a second input. As a result, the endovascular device 1200 may be moved into a second configuration that is different from the first configuration. For example, a series of inputs may be received in the course of a procedure being performed with endovascular device 1200. In response to each received input, the processor of the control device may move the endovascular device 1200 into a different configuration, thus performing the desired procedure.

In some embodiments, the at least one processor of the control device (e.g., controller 2414) may be configured to actuate the device movement mechanism 2412 to move a first movable portion and a second movable portion of the endovascular device 1200 in the same direction. According to embodiments including the expandable mesh device shown in FIGS. 4A-4D, the device movement mechanism 2412 may be configured to move both the control wire 4230 and the elongated shaft 4220 of the expandable mesh device. The control wire 4230 may be secured with respect to the mesh's distal end 4212, while the shaft 4214 may be secured with respect to the mesh's proximal end 4214. As a result, the two ends of the mesh may be movable and therefore, may be manipulated to achieve a desired shape and/or configuration of the mesh 4210, under control of the device movement mechanism 2412. In some embodiments, device movement mechanism 2412 may move the distal end 4212 and proximal end 4214 in the same direction (e.g., may move both in a distal direction during advancement of the mesh 4210 to a treatment site). Additionally, or alternatively, the at least one processor of the control device (e.g., controller 2414) may be configured to actuate the device movement mechanism 2412 to move the first movable portion of the endovascular device in a first direction and move the second movable portion of the endovascular device in a second direction that is opposite the first direction. For example, device movement mechanism 2412 may expand mesh 4210 and may moving distal end 4212 in a proximal direction while also moving proximal end 4214 in a distal direction. Additionally, or alternatively, the at least one processor of the control device (e.g., controller 2414) may be configured to actuate the device movement mechanism 2412 to move at least one movable portion of the endovascular device while another portion of the endovascular device remains stationary relative to the control device. For example, the device movement mechanism 2412 may also be configured to move one of the mesh's distal end 4212 and proximal end 4214, while the other end of the mesh remains stationary. By this alternative technique, mesh 4210 may be expanded and/or contracted.

In some embodiments, the at least one processor of the control device (e.g., controller 2414) may be configured to, in response to a third input, actuate the device movement mechanism 2412 to move the core wire 4230 of the endovascular device to exert a pulsatile force on an inner surface of a body structure (e.g., a rhythmic massaging motion). For example, the pulsatile movement may be activated by button 3434 of control device 1400a, shown in FIG. 3A. The pulsatile movement may be optimized to improve the integration between endovascular device 1200 and a clot 4012 or other obstruction by ranging the motion of a mesh 4210 of the endovascular device from a first position creating a maximal radial force to a second position creating a lower force. The movement frequency of the pulsatile movement described above may range from 0.1 Hz to 5 Hz.

In some embodiments, the at least one processor of the control device (e.g., controller 2414) may be configured to, in response to a fourth input, actuate the device movement mechanism 2412 to move the core wire (e.g., control wire 4230) in the second direction (e.g., a distal direction), thereby causing the mesh 4210 to contract, and to subsequently retract the endovascular device from the body structure (which may include a blood vessel). For example, button 3436 on control device 1400 (see FIGS. 3A and 3B) may bring the endovascular device 1200 back to a retrieval position, where the retrieval position may include the closing or radial contraction of a mesh 4210 of the endovascular device or, in alternative embodiments, straightening of a guide wire (see FIGS. 5A-5B) or straightening of a catheter (see FIG. 6) of the endovascular device. Control device 1400 may be configured to pull (or remove) the endovascular device 1200 into a delivery catheter and/or out of the body of the patient.

According to embodiments including the guide wire device of FIGS. 5A-5B, the at least one processor of the control device (e.g., controller 2414) may be configured to, in response to the first input, actuate the device movement mechanism 2412 to move the core wire of the guide wire device (e.g., a control wire, not shown) in a first direction (e.g., a proximal direction, to the right in FIGS. 5A-5B) while holding the elongated shaft 5220 of the guide wire device stationary, thereby causing the distal portion 5202 of the guide wire device to bend from a straightened configuration to a first bent configuration. Further, in response to a second input, the at least one processor of the control device (e.g., controller 2414) may be configured to actuate the device movement mechanism 2412 to move the core wire of the guide wire device in a second direction opposite to the first direction (e.g., a distal direction, or to the left in FIGS. 5A-5B) while holding the elongated shaft 5220 of the guide wire device stationary, thereby causing the distal portion 5202 of the guide wire device to bend from the first bent configuration to one of the straightened configuration or a second bent configuration.

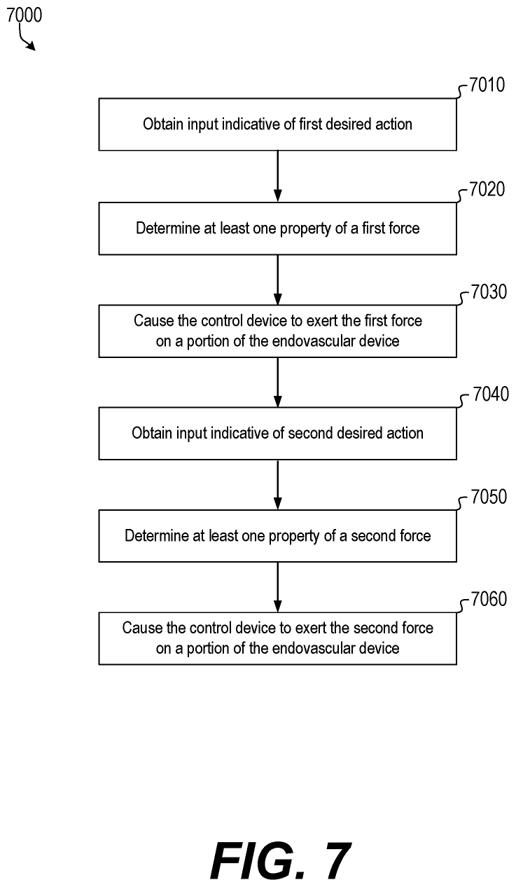

FIG. 7 is a flowchart illustrating an example of a process 7000 for digitally controlling an endovascular device, consistent with disclosed embodiments. Process 7000 is provided by way of example, and a person of ordinary skill would appreciate various other processes for digital control of an endovascular device consistent with this disclosure. At step 7010, process 7000 may include obtaining an input indicative of a first desired action of an endovascular device within a body structure of a patient. Examples of an endovascular device include endovascular devices comprising the expandable mesh 4210 of FIGS. 4A-4D, the guide wire comprising at least one bendable section of FIGS. 5A-5B, and the catheters comprising at least one bendable section of FIG. 6. At step 7020, process 7000 may include determining at least one property of a first force based on the obtained input. At step 7030, process 7000 may include causing a control device of the endovascular device to exert the first force on a first portion of the endovascular device based on the determined at least one property. In some embodiments, the first portion of the endovascular device may be positioned outside the body of the patient. Exertion of the first force may cause a second portion of the endovascular device (e.g., a portion within the body of the patient) to execute the first desired action within the body structure. At step 7040, process 7000 may include obtaining a second input indicative of a second desired action of the endovascular device within the body structure of the patient. At step 7050, process 7000 may include determining at least one property of a second force based on the obtained second input. At step 7060, process 7000 may include causing the control device of the endovascular device to exert the second force on a first portion of the endovascular device and/or on a third portion of the endovascular device, based on the determined at least one property of the second force. Exertion of the second force may cause a second portion or a fourth portion of the endovascular device (e.g., a portion within the body of the patient) to execute the second desired action within the body structure.

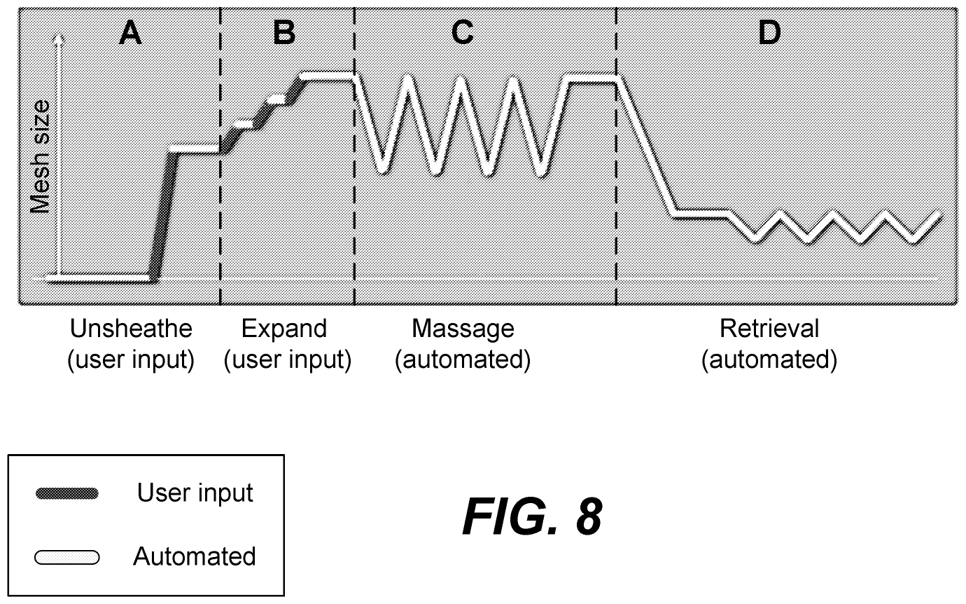

FIG. 8 depicts a graph showing an example of changes over time in the size of an expandable mesh 4210 of an endovascular device 1200, during a partially automated treatment of a body structure with the endovascular device 1200, consistent with disclosed embodiments. Partial automation may include part of the operation being executed based on input received from the user (e.g., via one of the control device configurations depicted in FIGS. 3A and 3B) and part of the operation being automated (i.e., executed by the control device 1400 based on pre-programmed instructions, without user input). For example, in embodiments in which the endovascular device is a device with an expandable mesh 4210, mesh size may increase during an unsheathing phase of the endovascular device 1200, where the unsheathing may be executed in response to input from the user. The size of mesh 4210 may increase in a step-wise fashion during an expansion phase of the endovascular device 1200, where the expansion may be executed in response to input from the user. The mesh size may fluctuate during a “massage,” or pulsatile or periodic, phase that is controlled by automation. Furthermore, mesh size may decrease and fluctuate during a retrieval phase that is controlled by automation. In FIG. 8, portions of the line graph in lighter gray indicate that the action is executed in response to a user input, whereas portions of the line graph in black indicate that the action is automated.

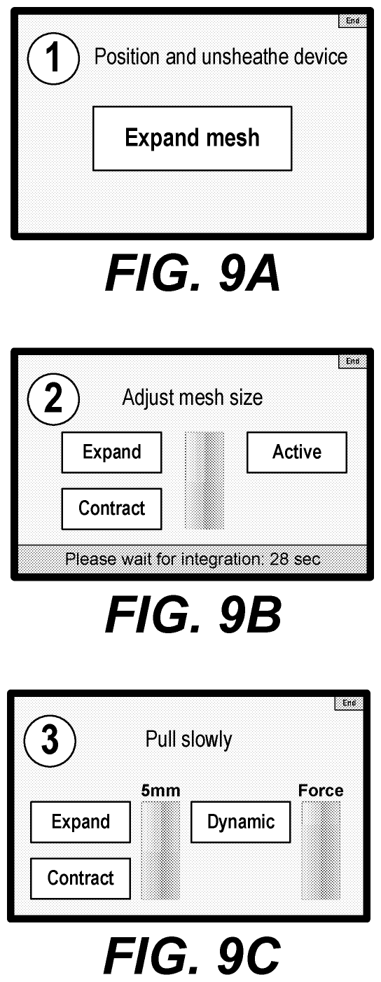

FIGS. 9A-9C illustrate examples of a graphical user interface (GUI) for display on a computing device of endovascular treatment system 1000, such as peripheral device 1800. The GUIs shown in FIGS. 9A-9C may include user input fields (e.g., buttons) that instruct the control device 1400 to execute one or more specific operations with the endovascular device 1200. For example, in embodiments in which the endovascular device is a device with an expandable mesh 4210, the buttons on the GUIs may cause the control device 1400 to expand the mesh, relax the mesh, operate a massage (pulsatile or periodic) mechanism, and go (return) to a retrieval position (e.g., with collapsed mesh). Operation of the GUIs shown in FIGS. 9A-9C may also enable the user to select the diameter of a vessel or the type of clot or other obstruction or narrowing to be treated with endovascular device 1200, an option for automation, or an option to insert the maximum magnitude of opening the endovascular device 1200. An option for automation may include the user pushing an interactive key or button for automatic function of the endovascular device 1200.

As shown in FIG. 9A, a first interactive display may include an input (e.g., a button) for the user to instruct control device 1400 to position and unsheathe the endovascular device 1200. The first interactive display of FIG. 9A may also include an interactive selection to expand the mesh 4210 of the endovascular device. As shown in FIG. 9B, a second interactive display may direct the control device 1400 to adjust the mesh size of the endovascular device 1200. The second interactive display may include interactive selections to adjust the mesh size such as expand or relax the mesh 4210. The second interactive display may also display whether the endovascular device 1200 is active. Furthermore, the second interactive display of FIG. 9B may display an estimated duration of time until integration. As shown in FIG. 9C, a third interactive display may direct the control device 1400 to remove the endovascular device 1200 from the patient's body. The third interactive display of FIG. 9C may include interactive selections to adjust the mesh size such as expand or relax the mesh 4210 of the endovascular device 1200. The third interactive display may also display the mode that the endovascular device 1200 is in. Furthermore, the third interactive display may display a force measured by a sensor 1600.

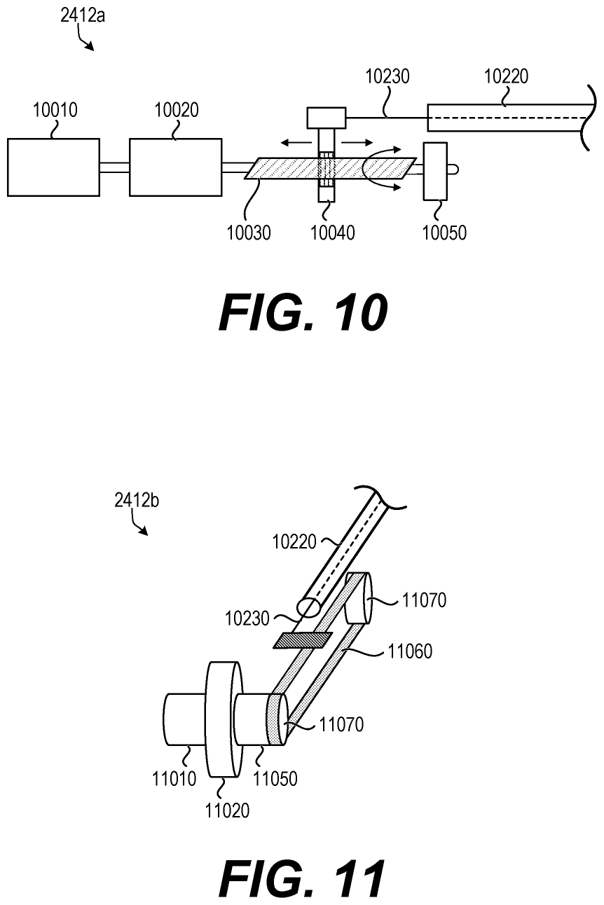

FIG. 10 depicts an embodiment of a device movement mechanism 2412a of control device 1400, consistent with disclosed embodiments. Device movement mechanism 2412a may include a motor 10010, a gearbox 10020, a helical or screw gear 10030, a screw gear load 10040, and an encoder 10050. Motor 10010 may be powered by power source 2416 (see FIG. 2) and controlled by signals received from controller 2414. Motor 10010 may be operably connected to screw gear 10030 by gearbox 10020. A first end of screw gear load 10040 may be secured to a core wire 10230 of endovascular device 1200 (e.g., core wire 4230 of FIGS. 4A-4D), while a second end of screw gear load 10040 may include teeth that engage the teeth of screw gear 10030. Motor 10010 may drive rotation of screw gear 10030, which may translate to longitudinal movement of screw gear load 10040 (to the left and right in FIG. 10). Longitudinal movement of screw gear load 10040 may move core wire 10230 relative to a shaft 10220 of endovascular device 1200 (e.g., elongated shaft 4220 of FIGS. 4A-4D, elongated shaft 5220 of FIGS. 5A-5B, or elongated sheath 6220 of FIG. 6). Shaft 10220 may be secured to control device 1400 or a portion thereof, thus allowing the movement of core wire 10230 to translate to execution of the desired action of endovascular device 1200. Encoder 10050 may sense the rotational speed and direction of screw gear 10030, converting the detected rotation to an electrical signal that may be output to controller 2414 as a form of feedback relating to the detected speed and direction. Controller 2414 may receive the feedback signal from encoder 10050 and may control the operation of motor 10010 based on the signal (e.g., by increasing or decreasing the speed of motor 10010, by changing the gear of gearbox 10020 connecting motor 10010 to screw gear 10030, or by starting or stopping the drive of motor 10010).

FIG. 11 depicts another embodiment of a device movement mechanism 2412b of control device 1400, consistent with disclosed embodiments. Device movement mechanism 2412b may include a motor 11010 and gearbox 11020 configured to drive rotation of a pulley 11070, which may drive a belt 11060 connected to core wire 10230. Thus, core wire 10230 may be moved relative to endovascular device shaft 10220 via the core wire's connection to the drive belt 11060 (as discussed in detail above). Device movement mechanism 2412b may similarly include an encoder 11050, which may provide feedback to controller 2414 relating to the speed and rotational direction of drive belt 11060 (as discussed above).

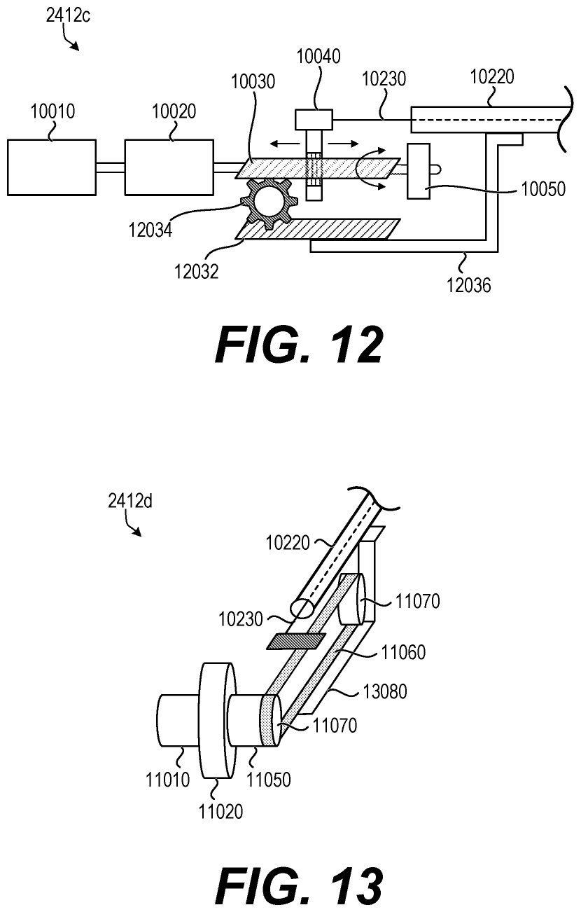

FIG. 12 depicts another embodiment of a device movement mechanism 2412c of control device 1400, consistent with disclosed embodiments. Device movement mechanism 2412c may be similarly configured as device movement mechanism 2412a of FIG. 10, with the addition of a mechanism for moving endovascular device shaft 10220 (in addition to movement of core wire 10230). Device movement mechanism 2412c may include a second screw gear 12032 and a gear wheel 12034 connecting screw gear 10030 to second screw gear 12032. Second screw gear 12032 may be connected to endovascular device shaft 10220 via a rigid connector 12036, which may move the shaft 10220 in response to a corresponding movement of second screw gear 12032. When motor 10010 drives rotation of screw gear 10030 and the corresponding movement of core wire 10230, gear wheel 12034 may translate the rotation of screw gear 10030 to a corresponding axial movement of second screw gear 12032 (to the left and right in FIG. 12). In some embodiments, second screw gear 12032 may translate in the opposite direction of screw gear load 10040, with the magnitude of their respective translations depending upon the configuration of gear wheel 12034. For example, in cases when motor 10010 drives screw gear load 10040 to translate in a distal direction (to the right in FIG. 12), second screw gear 12032 may be driven to translate in a proximal direction (to the left in FIG. 12), and vice versa. Due to the connection between second screw gear 12032 and endovascular device shaft 10220 via rigid connector 12036, translation of second screw gear 12032 may cause a similar translation of shaft 10220. As a result, core wire 10230 and shaft 10220 may both be moved, in opposite directions, by motor 10010. In some embodiments including an expandable mesh 4210, core wire 10230 may be connected to mesh distal end 4212 (or to a distal tip 4240, which may be secured relative to mesh distal end 4212), while shaft 10220 may be connected to mesh proximal end 4214. Thus, when device movement mechanism 2412c is driven by motor 10010, the distal end 4212 and proximal end 4214 of the mesh may be driven together or apart, causing expansion or contraction of mesh 4210, respectively. In some embodiments, the ratio of movement between screw gear load 10040 and second screw gear 12032 (and thus, the ratio of movement between core wire 10230 and shaft 10220) may depend upon the selection of gear wheel 12034. In some embodiments, control device controller 2414 may be configured to adjust the ratio of movement between core wire 10230 and shaft 10220 by changing gear wheel 12034 to a different gear wheel that changes the ratio of axial movement between screw gear load 10040 and second screw gear 12032 (thus changing the ratio of respective movements of core wire 10230 and shaft 10220).

FIG. 13 depicts another embodiment of a device movement mechanism 2412d of control device 1400, consistent with disclosed embodiments. Device movement mechanism 2412d may be similarly configured as device movement mechanism 2412b of FIG. 11, with the addition of a mechanism for moving endovascular device shaft 10220 (in addition to movement of core wire 10230). Device movement mechanism 2412c may include a rigid connector 13080 that connects drive belt 11060 to endovascular device shaft 10220. Thus, when motor 11010 drives the belt 11060 to cause movement of core wire 10230, rigid connector 13080 may translate the rotation of belt 11060 to endovascular device shaft 10220, thus causing equal and opposite movements of core wire 10230 and shaft 10220, which may occur simultaneously.

Consistent with disclosed embodiments, a control device 1400 may be provided for controlling an endovascular device 1200, the control device configured to control movement of multiple movable portions of endovascular device 1200. For example, control device 1400 may include a first mechanism for controlling a shaft 10220 of the endovascular device, as well as a second mechanism for controlling a core wire 10230 of the endovascular device (e.g., a control wire). Examples of the first mechanism may include rigid connector 12036 of FIG. 12 and rigid connector 13080 of FIG. 13. Examples of the second mechanism may include screw gear load 10040 of FIGS. 10 and 12 and drive belt 11060 of FIGS. 11 and 13. In disclosed embodiments, control device 1400 may be configured to actuate the first mechanism and second mechanism in response to a first input from a user. For example, the input may be obtained from a button or other user input mechanism of control device 1400 (e.g., a button depicted in FIG. 3A or 3B). In response to the first input, control device 1400 may actuate the first and second mechanisms to move the shaft 10220 of the endovascular device a first distance in a first direction (e.g., a distal direction) and move the core wire 10230 of the endovascular device a second distance in a second direction (e.g., a distal direction) that is opposite the first direction. The first direction and second direction may be opposites; for example, when motor 10010 drives movement of core wire 10230 in a distal direction, motor 10010 may also drive movement of shaft 10220 in a proximal direction, and vice versa. In some embodiments, the first mechanism and second mechanism of control device 1400 may be configured to move the shaft 10220 and core wire 10230 simultaneously, such as in the embodiments shown in FIGS. 12 and 13. Additionally, or alternatively, the first mechanism and second mechanism of control device 1400 may be configured to move the shaft 10220 and core wire 10230 sequentially (i.e., with one movement occurring before the other movement). In disclosed embodiments, control device 1400 may be configured to actuate the first and second mechanisms in response to a single input from the user (e.g., due to the user pressing a single button on the user interface of control device 1400, such as a button depicted in FIGS. 3A-3B).

In some embodiments, the first distance (i.e., distance of movement of shaft 10220) may be equal in magnitude to the second distance (i.e., distance of movement of core wire 10230). Additionally, or alternatively, one of the first distance and second distance may be larger than the other. Additionally, or alternatively, controller 2414 of the control device 1400 may be configured to controllably adjust the ratio between a displacement of the shaft 10220 (e.g., the first distance) and a corresponding displacement of the core wire 10230 (e.g., the second distance). For example, the control device 1400 may change gear wheel 12034 in the embodiment of FIG. 12, which may adjust the ratio between the distance traveled by core wire 10230 and the distance traveled by shaft 10220 when both the core wire and shaft are driven by motor 10010. As a result, the at least one processor of the control device 1400 (e.g., controller 2414) may be configured to adjust the ratio between a displacement of a first movable portion of the endovascular device (e.g., mesh proximal end 4214) by the device movement mechanism 2412 and a corresponding displacement of a second movable portion of the endovascular device (e.g., mesh distal end 4212) by the device movement mechanism 2412.

In some embodiments, the control device 1400 may additionally be configured to actuate the first and second mechanisms in response to a second input from the user, such as during a procedure being performed with endovascular device 1200. In response to the second input, control device 1400 may actuate the first and second mechanisms to move the shaft 10220 a third distance in the first direction (e.g., a proximal direction) and move the core wire 10220 a fourth distance in the second direction (e.g., a distal direction), the third distance differing from the first distance and the fourth distance differing from the second distance. For example, the second input may indicate a smaller scale of movement than the first input, such that the first and second distances may be larger than the third and fourth distances, respectively. In some embodiments, a ratio between the second distance and the first distance may be substantially the same as the ratio between the fourth distance and the third distance (such as when the same gear wheel 12034 is used). In alternative embodiments, each input from the user (i.e., the first input and second input) may be associated with a respective magnitude. In such instances, the ratio between the third distance and the first distance may be substantially the same as the ratio between the magnitude associated with the second input and the magnitude associated with the first input. For example, the first input may indicate a larger scale of movement (i.e., a larger displacement of both the shaft 10220 and the core wire 10230) than the second input, such as respective magnitudes of 2 and 1 for the first input and second input, respectively. (This may occur, for example, due to the user holding an activation button 3430 twice as long for the first input than for the second input, which may correspond to a magnitude of “2” for the first input and magnitude of “1” for the second input.) The corresponding movements of the shaft 10220 in response to the first input and in response to the second input will accordingly have a 2:1 ratio. Similarly, the corresponding movements of the core wire 10230 in response to the first input and in response to the second input will also accordingly have a 2:1 ratio.

In disclosed embodiments, the control device 1400 may be configured to, in response to a third input from the user, actuate the first and second mechanisms to move the shaft 10220 of the endovascular device in the second direction (e.g., a proximal direction) and to release the core wire 10230 of the endovascular device. For example, the third input may indicate a complete contraction or “collapse” of the endovascular device 1200. By releasing core wire 10230 and moving the shaft 10220 proximally, the control device 1400 may move endovascular device 1200 (e.g., mesh segment 4210) to the most contracted and low-profile configuration. This may allow removal of the endovascular device 1200 from the treatment site (e.g., when button 3436 on the control device of FIGS. 3A-3B is pressed).

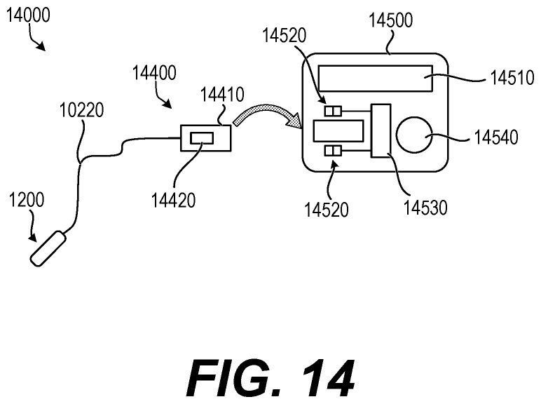

Consistent with disclosed embodiments, FIG. 14 depicts an endovascular treatment system 14000 configured to control an endovascular device 1200 with magnetic force. System 14000 may include a control device 14400, which may be configured to control an action of endovascular device 1200 at a treatment site within the body of a patient. Control device 14400 may include a control device body 14410, which may be configured as a portable, disposable, handheld device and which may be configured to be positioned outside the body of the patient. Control device 14400 may include a device movement mechanism configured to control at least one adjustable portion of the endovascular device 1200. Examples of the at least one adjustable portion may include expandable mesh 4210 of FIGS. 4A-4D, bendable segment 5202 of the guide wire shown in FIGS. 5A-5B, and deflectable segment 6202 of the catheter shown in FIG. 6. In embodiments including expandable mesh 4210, the endovascular device may be a stent retriever (e.g., a clot retrieval device) or may be an adjustable mesh device (e.g., configured to provide support to a blood vessel during treatment of aneurysm or configured to dilate a narrowed blood vessel). The at least one adjustable portion of the endovascular device 1200 may be configured for placement at the treatment site within the body of the patient, such as during a procedure being performed with the endovascular device. In some embodiments, the device movement mechanism of control device 14400 may be connected to shaft 4220 of the endovascular device 1200 and/or to a core wire 4230 of endovascular device 1200, and may be configured to move the shaft 4220 and/or core wire 4230 in order to cause movement of endovascular device 1200 (as discussed above).

Control device 14400 may include at least one magnet 14420 operably connected to the device movement mechanism. In some embodiments, the at least one magnet 14420 may include a single magnet or a plurality of magnets. In some embodiments, magnet 14420 may be fixed directly to the device movement mechanism. Alternatively, the at least one magnet 14420 may be connected to the device movement mechanism via a gear mechanism. The at least one magnet 14420 may be configured to be actuated by a second magnet 14520 of a user interface device 14500 (discussed further below), which may cause the at least one magnet 14420 and the device movement mechanism to control a movement of endovascular device 1200. For example, in response to a first actuation of the at least one magnet 14420, the device movement mechanism of control device 14400 may be configured to cause an action by the at least one adjustable portion of the endovascular device 1200, so that the endovascular device transitions into a first configuration at the treatment site. The action may include expansion or contraction of a mesh segment 4210, bending or straightening of a distal portion of the endovascular device (e.g., bendable segment 5202 of FIGS. 5A-5B or deflectable segment 6202 of FIG. 6), or inflation or deflation of a balloon device (not shown).

In response to a second actuation of the at least one magnet 14420, the device movement mechanism may be configured to cause a second action of the at least one adjustable portion of the endovascular device 1200, so that the endovascular device transitions into a second configuration at the treatment site, the second configuration being different from the first configuration. For example, control device 14400 may move endovascular device 1200 into the first configuration and into the second configuration as steps of a procedure performed at the treatment site using endovascular device 1200. Examples of the second configuration may include expansion or contraction of a mesh segment 4210 or bending or straightening of a distal portion of the endovascular device (e.g., bendable segment 5202 of FIGS. 5A-5B or deflectable segment 6202 of FIG. 6).

System 14000 may include a user interface device 14500, which a user may operate to control actions of endovascular device 1200 at the treatment site. In some embodiments, the user interface device 14500 is a portable, reusable, handheld device. User interface device 14500 may be placed in a sterile polyethylene bag (or in a similar sterilization pouch or peel pack), to keep sterility throughout the endovascular procedure. User interface device 14500 may include an input mechanism 14540 configured to receive input from a user. For example, input mechanism 14540 may include a first input structure, such as at least one of a button, a keyboard, a computer mouse, a lever, a joystick, or a touch screen. User interface device 14500 may also include at least a second magnet 14520 configured to actuate the at least one magnet 14420 of control device 14400 based on the input received from the user via input mechanism 14540. The at least one second magnet 14520 of user interface device 14500 may include a single magnet or a plurality of magnets.

In some embodiments, the user interface device 14500 may include an actuator 14530 configured to control movement of the second magnet 14520. In some embodiments, actuator 14530 may include a motor. Additionally, or alternatively, actuator 14530 may be connected to the second magnet 14520 by a mechanical connector, a gear mechanism, or by any other suitable type of connection mechanism. User interface device 14500 may also include at least one processor (not shown), which may be configured to cause the actuator 14530 to control movement of the second magnet 14520, such as based on at least one of a user input received by the input mechanism 14540, data of at least one medical image captured during a procedure performed with the endovascular device (such as an image captured by a peripheral imaging device 1800 shown in FIG. 1 or by an additional imaging device), an output from at least one sensor (such as output from sensor 1600 shown in FIG. 1), or computer-executable instructions for treatment with the endovascular device 1200, stored in a memory. In some embodiments, the at least one sensor (e.g., sensor 1600 of FIG. 1) may include a force meter configured to measure a force exerted by control device 14400 or by a user to remove the endovascular device 1200 from a body structure. In such cases, the at least one processor of user interface device 14500 may be configured to obtain a force measurement signal from the force meter and based on the force measurement signal, control movement of the second magnet 14520 by the actuator 14530 so as to actuate the at least one magnet 14420 of the control device. For example, the at least one processor may cause actuation of the at least one magnet 14420 to reduce the magnitude of force being exerted on the body structure by endovascular device 1200. Additionally, or alternatively, the at least one processor of user interface device 14500 may output an alert notification on display mechanism 14510 to alert a user when the magnitude of force being exerted during removal of endovascular device 1200 is too high.

In some embodiments, the at least one processor of user interface device 14500 may be configured to obtain an input indicative of a desired movement of the endovascular device 1200 at the treatment site; determine at least one property of a first force based on the input; and based on the determined at least one property, cause the actuator 14530 to exert the first force on the second magnet 14520 to actuate the at least one magnet 14420 of the control device 14400. In some embodiments, exertion of the first force on the second magnet 14520 may causes the device movement mechanism of control device 14400 (which may be connected to the at least one magnet 14420) to control the at least one adjustable portion of the endovascular device 1200 to execute the desired movement at the treatment site. The at least one property of the first force may include at least one of a magnitude of the first force, a time duration of the first force, or a direction of the first force. In the embodiment of FIG. 14, the at least one processor of user interface device 14500 may be configured to obtain an input indicative of a desired movement of the endovascular device, to determine at least one property of a first force based on the input, and to cause the actuator 14530 to exert the first force on the second magnet 14520 based on the determined at least one property in a manner consistent with the above discussion of the embodiment shown in FIG. 1.

In disclosed embodiments, user interface device 14500 may include at least one output mechanism for outputting one or more notifications to the user. Examples of the output mechanism may include visual display mechanism 14510 and an audio output mechanism. User interface device 14500 may also include a power source (not shown).

In some embodiments, endovascular treatment system 14000 may be configured to control actions of an endovascular device having an adjustable (e.g., expandable) mesh, such as expandable mesh 4210. In disclosed embodiments, the device movement mechanism of control device 14400 (which may be connected to the at least one magnet 14420) may be connected to a core wire 4230 of endovascular device 1200. The shaft 4220 may be connected to, or secured relative to, the control device 14400. In some embodiments, the device movement mechanism of control device 14400 may be configured to move the core wire in a first direction (e.g., a proximal direction) in response to a first actuation of the at least one magnet 14420 of the control device 14400. This movement of the core wire 4230 may cause an expandable mesh 4210 of endovascular device 1200 to expand. Additionally, or alternatively, the device movement mechanism of control device 14400 may be configured to move the core wire 4230 in a second direction (e.g., a distal direction) in response to a second actuation of the at least one magnet 14420 of the control device 14400, thereby causing the mesh 4210 to contract.

Additionally, or alternatively, the device movement mechanism of control device 14400 may be configured to move the core wire of endovascular device 1200 to exert a pulsatile force on an inner surface of a body structure at the treatment site in response to a third actuation of the at least one magnet 14420 of the control device 14400. Exertion of the pulsatile force in the embodiment of FIG. 14 may be achieved in a similar manner to the above discussion of exertion of the pulsatile force with respect to the embodiment of FIGS. 3A, 3BB and 8. Additionally, or alternatively, the device movement mechanism of control device 14400 may be configured to move the core wire 4230 in a second direction (e.g., a distal direction) in response to a fourth actuation of the at least one magnet 14420 of the control device, which may cause the mesh 4210 to contract. The device movement mechanism may also be configured to subsequently withdraw the endovascular device 1200 with the contracted mesh 4210 from the body structure and/or from the body of the patient.

In disclosed embodiments, control device 14400 may be configured to cause expansion and contraction of the mesh 4210 based on user input obtained via the input mechanism 14540 of the user interface device 14500. The obtained user input may specify at least one of the diameter of a body structure at the treatment site, a type of narrowing or obstruction (e.g., a blood clot, vasospasm, or plaque) to be treated with the endovascular device 1200, a type of clot to be treated with the endovascular device 1200, a desired expansion diameter or shape of the expandable mesh 4210, an instruction to expand or contract the mesh 4210 to a specific configuration, or a desired expansion and/or contraction time (e.g., pulsatile or massage movement). Control device 14400 may be configured to control movement of the expandable mesh 4210 to achieve the configuration of the endovascular device specified by the user input.

In some embodiments, endovascular treatment system 14000 may be configured to control actions of an endovascular device being a guide wire (e.g., endovascular device 1200b of FIGS. 5A-5B). Control device 14400 may be configured to control bending and/or straightening of at least one distal portion 5202 of the guide wire device by manipulating a core wire of the guide wire device (not shown). The core wire may be configured as a control wire. The distal portion may include the distal tip.

In response to the first actuation of the at least one magnet 14420 of the control device 14400, the device movement mechanism may be configured to move the core wire (e.g., control wire) of the guide wire device in a first direction (e.g., a proximal direction), while holding the elongated shaft 5220 of the guide wire device stationary, thereby causing the bendable distal portion 5202 of the guide wire device to bend from a straightened configuration to a first bent configuration. In response to a second actuation of the at least one magnet 14420 of the control device 14400, the device movement mechanism may be configured to move the core wire of the guide wire device in a second direction (e.g., a distal direction) opposite to the first direction, while holding the elongated shaft 5220 of the guide wire device stationary, thereby causing the distal portion 5202 of the guide wire device to bend from the first bent configuration to one of the straightened configuration or a second bent configuration.

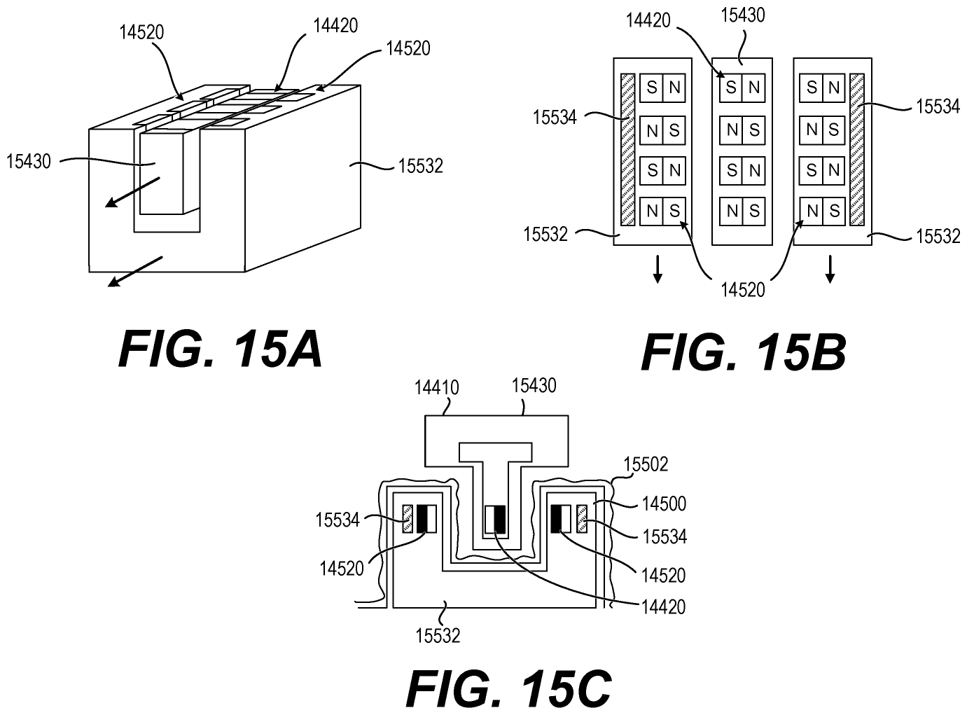

FIG. 15A-C depict an isometric view, top view, and front view, respectively, of a cross-section of magnets according to some embodiments of a device movement mechanism of endovascular treatment system 14000. Magnetic movement housing 15430 may house a first at least one magnet 14420. The first at least one magnet 14420 may be operably connected to the device movement mechanism, as shown in FIG. 14. The first at least one magnet 14420 may be configured to be actuated by a second at least one magnet 14520 of user interface device 14500, as shown in FIG. 14. The second at least one magnet 14520 may be included in a moving cart 15532. The moving cart 15532 may be connected to the actuator 14530 of the user interface device 14500 shown in FIG. 14. In response to a first actuation of the first at least one magnet 14420, the device movement mechanism of control device 14400 may be configured to cause an action by the at least one adjustable portion of the endovascular device 1200, as discussed above with reference to FIG. 14. Additionally, or alternatively, the device movement mechanism of control device 14400 may be configured to cause a movement of the endovascular device 1200 (e.g., in a distal or proximal direction), as discussed above.

The first at least one magnet 14420 and second at least one magnet 14520 may form multiple sets of magnets. For example, as shown in the embodiment depicted in FIGS. 15A-15B, the first at least one magnet 14420 and second at least one magnet 14520 may form 4 sets of magnets. It is contemplated that the first at least one magnet 14420 and second at least one magnet 14520 may form any number of sets of magnets, such as but not limited to, 2 sets, 3 sets, 4 sets, 5 sets, 6 sets, 7 sets, 8 sets, 9 sets, 10 sets, or more of magnets.

As shown in the embodiment depicted in FIG. 15B, which illustrates a top view of a cross-section of a device movement mechanism, the first at least one magnet 14420 and second at least one magnet 14520 may be arranged such that for each set of magnets, opposite poles (N for north and S for south) are facing each other. In some embodiments, ferromagnetic material 15534 may be disposed on an outer edge of the second at least one magnet 14520 to close the magnetic field lines and/or prevent generation of undesirable magnetic force.

As shown in FIG. 15C, magnetic movement housing 15430 may be disposed in control device body 14410 of control device 14400. Control device body 14410 may be connected to a core wire (not shown) of an endovascular device. In some embodiments, control device body 14410 may be connected to other types of endovascular devices. In some embodiments, a sterile bag 15502 may partially or fully encapsulate the user interface device 14500, separating a non-sterile user interface device 14500 from a sterile endovascular device (not shown) and sterile control device body 14410.

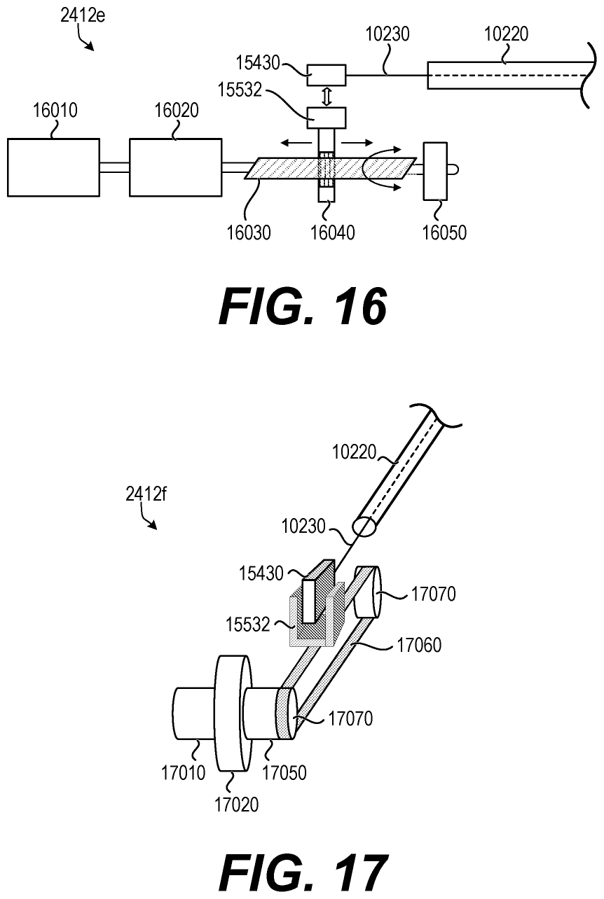

FIG. 16 depicts an embodiment of a device movement mechanism 2412e of endovascular treatment system 14000, consistent with disclosed embodiments. Device movement mechanism 2412e may be similarly configured as device movement mechanism 2412a of FIG. 10 and/or device movement mechanism 2412c of FIG. 12. Device movement mechanism 2412e may include a motor 16010, a gearbox 16020, a screw gear 16030, a screw gear load 16040, and an encoder 16050. Device movement mechanism 2412e may also include magnetic movement housing 15430 and moving cart 15532. Magnetic movement housing 15430 may include first at least one magnet 14420. Moving cart 15532 may include second at least one magnet 14520. The first at least one magnet 14420 may be configured to be actuated by second at least one magnet 14520 of a user interface device 14500, as discussed above. In some embodiments, the first at least one magnet 14420 may be connected to core wire 10230. Longitudinal movement of screw gear load 10040 may result in movement of the first at least one magnet 14520 and magnetic movement housing 15430, and correspondingly may move core wire 10230 relative to a shaft 10220 of endovascular device 1200 (e.g., elongated shaft 4220 of FIGS. 4A-4D, elongated shaft 5220 of FIGS. 5A-5B, or elongated sheath 6220 of FIG. 6) Shaft 10220 may be secured to control device 14410 or a portion thereof, thus allowing the movement of core wire 10230 to translate to execution of the desired action of endovascular device 1200. According to one embodiment, the magnetic movement housing 15430 may also be connected to endovascular device shaft 10220 via a rigid connector 10230, which may move endovascular device shaft 10220 in response to a corresponding movement of the first at least one magnet 14520 and magnetic movement housing 15430.

FIG. 17 depicts an embodiment of a device movement mechanism 2412f of endovascular treatment system 14000, consistent with disclosed embodiments. Device movement mechanism 2412f may be similarly configured as device movement mechanism 2412b of FIG. 11 and/or device movement mechanism 2412d of FIG. 13. Device movement mechanism 2412f may include a motor 17010, a gearbox 17020, a belt 17060, a pulley 17070, and an encoder 17050. Device movement mechanism 2412f may also include magnetic movement housing 15430 and moving cart 15532. Magnetic movement housing 15430 may include the first at least one magnet 14420. Moving cart 15532 may include the second at least one magnet 14520. The first at least one magnet 14420 may be configured to be actuated by second at least one magnet 14520 of a user interface device 14500, as discussed above.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. While certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps and/or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Throughout this application, various embodiments of the present disclosure may be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numeric values within that range. For example, description of a range such as from 1 to 6 should be considered to include subranges such as from 1 to 3, from 1 to 4, from 1 to 5, from 2 to 4, from 2 to 6, from 3 to 6, and so forth, as well as individual numbers within that range, for example, 1, 2, 3, 4, 5, and 6. This applies regardless of the breadth of the range.

Other embodiments will be apparent from consideration of the specification and practice of the embodiments disclosed herein. It is intended that the specification and examples be considered as example only, with a true scope and spirit of the disclosed embodiments being indicated by the following claims.

1. A system for digital control of an endovascular device, the system comprising:
at least one processor configured to:
obtain an input indicative of a first desired action of an endovascular device within a body structure of a patient;
determine at least one property of a first force based on the input; and
based on the determined at least one property, cause a control device of the endovascular device to exert the first force on a first portion of the endovascular device, the first portion of the endovascular device positioned outside the body of the patient, wherein exertion of the first force causes a second portion of the endovascular device to execute the first desired action within the body structure.

2. The system of claim 1, wherein the input includes at least one of
an input from a user performing a procedure with the endovascular device;
first data of at least one medical image captured prior to or during a procedure performed with the endovascular device; or
second data derived from at least one sensor output.

3. The system of claim 1, wherein the input from the user is obtained from at least one of
a control handle operably connected to the at least one processor, or
a device including at least a second processor.

4.-23. (canceled)

24. A control device for controlling movement of an endovascular device, the control device being configured to be positioned outside a body of a patient, the control device comprising:
an input mechanism configured to receive input from a user;
a device movement mechanism configured to control at least one movable portion of the endovascular device, the movable portion of the endovascular device configured for placement within the body of the patient; and
at least one processor configured to:
in response to a first input, actuate the device movement mechanism to move the at least one movable portion of the endovascular device, so that the endovascular device is moved into a first configuration.

25. The control device of claim 24, wherein the at least one processor is configured to:
in response to a second input, actuate the device movement mechanism to move the at least one movable portion of the endovascular device, so that the endovascular device is moved into a second configuration that is different from the first configuration.

26. The control device of claim 24, wherein the at least one processor is configured to actuate the device movement mechanism to do at least one of
move a first movable portion and a second movable portion of the endovascular device in the same direction;
move the first movable portion of the endovascular device in a first direction and move the second movable portion of the endovascular device in a second direction that is opposite the first direction; or
move at least one movable portion of the endovascular device while another portion of the endovascular device remains stationary relative to the control device.

27. The control device of claim 24, wherein at least one of the first input or the second input is a user input received via the input mechanism.

28. The control device of claim 24,
wherein the endovascular device comprises an expandable mesh and a core wire fixed relative to a distal end of the expandable mesh, and
wherein the control device is configured to actuate the core wire to cause expansion and/or contraction of the expandable mesh.

29. The control device of claim 28, wherein the at least one processor is configured to:
in response to the first input, actuate the device movement mechanism to move the core wire of the endovascular device in a first direction, thereby causing the mesh to expand;
and/or in response to a second input, actuate the device movement mechanism to move the core wire in a second direction that is opposite the first direction, thereby causing the endovascular mesh to contract.

30.-49. (canceled)

50. An endovascular treatment system, comprising:
an endovascular device configured for controllable movement at a treatment site within the body of a patient, the endovascular device including at least one movable portion; and
a control device for controlling the endovascular device, the control device comprising:
an input mechanism configured to receive input from a user;
a device movement mechanism configured to control the at least one movable portion of the endovascular device; and
at least one processor configured to:
in response to a first input, actuate the device movement mechanism to move the at least one movable portion of the endovascular device, so that the endovascular device is moved into a first configuration at the treatment site.

51. The endovascular treatment system of claim 50, wherein the at least one processor is configured to:
in response to a second input, actuate the device movement mechanism to move the at least one movable portion of the endovascular device, so that the endovascular device is moved into a second configuration at the treatment site, the second configuration being different from the first configuration.

52. The endovascular treatment system of claim 50, wherein the at least one processor of the control device is configured to adjust the ratio between a displacement of a first movable portion of the endovascular device by the device movement mechanism and a corresponding displacement of a second movable portion of the endovascular device by the device movement mechanism.

53. The endovascular treatment system of claim 51, wherein the input mechanism comprises at least a first input structure for receiving the first input from the user and a second input structure for receiving the second input from the user, and wherein each of the first input structure and second input structure comprises at least one of a button, a keyboard, a computer mouse, a lever, a joystick, or a touch screen.

54. The endovascular treatment system of claim 50,
wherein the endovascular device comprises an expandable mesh and a core wire fixed with respect to a distal end of the expandable mesh, and
wherein the control device is configured to actuate the core wire to cause expansion and contraction of the expandable mesh.

55. The endovascular treatment system of claim 54, wherein the at least one processor of the control device is configured to:
in response to the first input, actuate the device movement mechanism to move the core wire of the endovascular device in a first direction, thereby causing the mesh to expand; and/or
in response to a second input, actuate the device movement mechanism to move the core wire in a second direction, thereby causing the mesh to contract.

56. The endovascular treatment system of claim 54 of 55, wherein the at least one processor of the control device is configured to:
in response to a third input, actuate the device movement mechanism to move the core wire of the device to exert a pulsatile force on an inner surface of the body structure; and/or
in response to a fourth input, actuate the device movement mechanism to move the core wire in the second direction, thereby causing the mesh to contract, and to subsequently retract the device from the body structure.

57. The endovascular treatment system of claim 54, wherein the at least one processor of the control device is configured to cause expansion and contraction of the expandable mesh based on at least one of
a user input via the input mechanism;
a signal from at least one sensor;
a signal from a processor of a user interface device; or
computer-executable instructions for treatment with the device, stored in a memory.

58. The endovascular treatment system of claim 50, further comprising at least one of
a force meter;
a user interface device; or
an image display device.

59. The endovascular treatment system of claim 58, wherein the at least one processor of the control device is configured to:
obtain a force measurement signal from the force meter; and
based on the force measurement signal, control a movement of the at least one movable portion of the endovascular device by the device movement mechanism.

60.-61. (canceled)

62. The endovascular treatment system of claim 50, wherein the device movement mechanism comprises at least one of a motor, an encoder, or a gear, and wherein the at least one of a motor, an encoder, or a gear of the device movement mechanism is configured to move the at least one movable portion of the endovascular device based on at least one of
a user input obtained via the input mechanism of the control device;
a user input obtained via a user interface device operably connected to the control device;
a force measurement signal obtained from a force meter; or
computer-executable instructions for treatment with the device, stored in a memory.

63. The endovascular treatment system of claim 50,
wherein the device movement mechanism of the control device comprises at least one magnet, and
wherein in response to a first actuation of the at least one magnet, the device movement mechanism is configured to cause movement of the at least one movable portion of the endovascular device, so that the endovascular device is moved into the first configuration.

64.-87. (canceled)
